(19) United States
(12) Patent Application Publication
(10) Pub. No.: US 2014/0241990 A1
(43) Pub. Date: Aug. 28, 2014

(54) METHODS OF USING ADENOSINE A1 RECEPTOR ACTIVATION FOR TREATING DEPRESSION

(71) Applicant: TUFTS UNIVERSITY, Boston, MA (US)

(72) Inventors: Philip G. Haydon, Boston, MA (US); Dustin J. Hines, Medford, MA (US); L. Ian Schmitt, Boston, MA (US)

(73) Assignee: TUFTS UNIVERSITY, Boston, MA (US)

(21) Appl. No.: 14/348,704

(22) PCT Filed: Sep. 28, 2012
(86) PCT No.: PCT/US2012/058126

§ 371 Date: Mar. 31, 2014

Related U.S. Application Data
(60) Provisional application No. 61/541,407, filed on Sep. 30, 2011.
(60) Provisional application No. 61/543,260, filed on Oct. 4, 2011.

(51) Int. Cl. A61K 31/7076; C07D 213/85; A61K 49/00

(52) U.S. Cl. CPC A61K 31/7076; A61K 49/00; C07D 213/85
USPC 424/9.2; 514/46; 514/344; 435/29

(57) ABSTRACT

Disclosed herein are methods for treating depression and depressive-like symptoms by administering a therapeutically effective amount of an adenosine 1 receptor agonist. Also disclosed herein are methods for identifying adenosine receptor agonists and the use of identified adenosine receptor agonists for treating diseases, disorders or conditions characterized by pathological sleep perturbations.

### BACKGROUND

Major depressive disorder (MDD) is a debilitating condition with a lifetime risk of ten percent. Current treatments take weeks for clinical efficacy, limiting the ability, for example, to bring instant relief needed with suicidal patients. One intervention that rapidly alleviates symptoms of depression is sleep deprivation; however, the mechanism remains unknown and there are serious side effects associated with sleep deprivation.

Sleep is an essential component of a healthy lifestyle, yet approximately 60% of people report disordered sleep patterns a few nights per week or more. Sleep disorders have been closely linked to a variety of other health and psychiatric conditions, raising sleep as a major health concern. In particular, a very close relationship exists between MDD and sleep disorders. In clinical samples, about three quarters of all depressed patients complain of difficulty either in initiation or in maintaining sleep, while epidemiologic data also suggest that hypersomnia, or excessively lengthy sleep episodes coupled with daytime sleepiness and frequent napping, is present in 10-40% of patients with various mood disorders.

Current pharmacological treatments for MDD take weeks for clinical efficacy, limiting the ability to bring instant relief to suicidal patients. Sleep deprivation is effective in alleviating MDD in approximately 60% of all depressed patients, and in about 70% of those patients diagnosed with “endogenous” depression. As there are significant limitations and concerns with current treatments for MDD and depressive disorders, there exists a need to develop faster-acting, more effective treatments with milder side effects.

### SUMMARY

Described herein are findings that demonstrate adenosine receptor agonists can be used to treat depression or depressive-like symptoms. Also described herein are methods for identifying adenosine receptor agonists, which can be used, for example, to treat or prevent diseases, disorders or conditions associated with pathological sleep perturbations, e.g., depression or depressive-like symptoms, sleep disorders in the elderly, Parkinson's disease, Alzheimer's disease, epilepsy, schizophrenia, and symptoms experienced by recovering alcoholics.

Traditional therapeutics for depression are limited by the delayed onset of beneficial effects. In contrast, the adenosine signaling pathway activated by sleep deprivation is rapidly beneficial and represents a novel strategy to bring rapid relief to, for example, suicidal patients where a delay in therapeutic benefits could be devastating. While adenosinergic pharmacology can alleviate depressive-like behaviors in mice, it could not become a therapeutic strategy for humans because of the systemic side effects resulting from the activation of A1Rs. That said, the results described herein provide a highly novel and innovative targeting glial specific receptors to influence adenosine signaling.

The mechanisms underlying the robust improvement observed in human depression patients following sleep deprivation were investigated. Described are data showing the beneficial effects of sleep deprivation on depressive like symptoms are controlled by the astrocyte-dependent signaling. In particular, A1R signaling is effective for producing the beneficial effects of sleep deprivation even in the absence of sleep deprivation. Findings described herein demonstrate the effective therapeutic utility of A1R agonists, e.g., CCPA, for treating depression and depressive-like symptoms.

In one embodiment, the disclosure is directed to a method of identifying a ligand that stimulates the cellular release of adenosine, comprising, a) introducing a test compound into a subject, tissue sample or cultured cells; and b) determining the release of adenosine, wherein an increase in released adenosine is indicative of the test compound's efficacy as a ligand that stimulates the cellular release of adenosine. In a particular embodiment, the extracellular adenosine concentration is determined using biosensor electrodes. In a particular embodiment, the test compound is an activity modulator of a receptor expressed in astrocytes. In a particular embodiment, the receptor and activity modulator are selected from the group of receptors and activity modulators of Table 1. In a particular embodiment, the test compound is introduced into the frontal cortex of the subject or a brain slice from the frontal cortex of the subject. In a particular embodiment, the method further comprises introducing a test compound into a subject, tissue sample or cultured cells, wherein dnSNARE is selectively expressed in astrocytes of the subject, tissue sample or cultured cells.

Disclosed herein are methods for treating or preventing a disease, disorder or condition characterized by pathological sleep perturbations, comprising administering to a subject a therapeutically effective amount of a compound identified as a ligand that stimulates the cellular release of adenosine by any of the methods described herein. Such compounds, e.g., identified A1R agonists and/or ligands that stimulate the release of adenosine in astrocytes, can be used, for example, for the manufacture of a medicament for the treatment of a disease, disorder or condition characterized by pathological sleep perturbations. The disease, disorder or condition can be, for example, depression or depressive-like symptoms, sleep disorders in the elderly, Parkinson's disease, Alzheimer's disease, epilepsy, schizophrenia and symptoms experienced by recovering alcoholics.

One embodiment is directed to a method of identifying a ligand that stimulates the cellular release of adenosine, comprising, a) contacting cultured astrocytes or an astrocyte-based cell line with a test compound; and b) determining the intracellular concentration of Ca²⁺, wherein an increase in intracellular Ca²⁺ is indicative of the test compound's efficacy as a ligand that stimulates the cellular release of adenosine. In a particular embodiment, the method further comprises introducing the test compound into astrocytes of an animal model, subject or tissue sample and determining the concentration of extracellular adenosine, wherein an increase in extracellular adenosine validates the efficacy of the test compound as a ligand that stimulates the release of adenosine. In a particular embodiment, the Ca²⁺ concentration is determined using a molecular marker indicative of Ca²⁺ concentration. In a particular embodiment, the molecular marker is a fluorescent marker. In a particular embodiment, the extracellular adenosine concentration is determined using biosensor electrodes. In a particular embodiment, the astrocyte-based cell line is a human astrocytoma cell line.

In one embodiment, the disclosure is directed to a method of treating or preventing depression comprising administering an effective amount of an adenosine receptor agonist, e.g., an adenosine A1 receptor agonist, to a subject. In a particular embodiment, the subject is diagnosed with or is at risk of developing depression. In a particular embodiment, the adenosine receptor agonist is an adenosine derivative. In a particular embodiment, the adenosine receptor agonist is a compound of formula I,

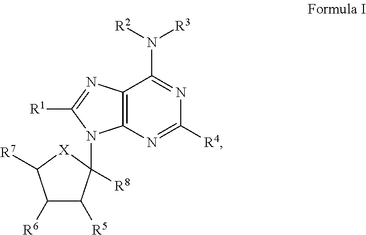

prodrugs, esters, or salts thereof wherein, X is O, S, NH, or CH₂; R¹, R², R³, R⁴, R⁵, R⁶, R⁷ and R⁸ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl or heterocyclyl, wherein each R¹, R², R³, R⁴, R⁵, R⁶, R⁷ and R⁸ are optionally substituted with one or more, the same or different, R⁹; R⁹ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁹ is optionally substituted with one or more, the same or different, R¹⁰; and R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In a particular embodiment, the R¹ is hydrogen. In a particular embodiment, R² is hydrogen, alkyl, carbocyclyl, aryl, or heterocyclyl, wherein R² is optionally substituted with one or more, the same or different R⁹. In a particular embodiment, R³ is hydrogen, alkyl, carbocyclyl, aryl, or heterocyclyl, wherein R³ optionally substituted with one or more, the same or different R⁹. In a particular embodiment, R⁴ is hydrogen or a halogen. In a particular embodiment, R⁵ is hydrogen, hydroxy, or alkoxy, and wherein R⁵ optionally substituted with one or more, the same or different R⁹. In a particular embodiment, R⁶ is hydrogen, hydroxy, or alkoxy, and wherein R⁶ optionally substituted with one or more, the same or different R⁹. In a particular embodiment, R⁷ is alkyl or formyl, and wherein R⁷ optionally substituted with one or more, the same or different R⁹. In a particular embodiment, R⁸ is hydrogen or alkyl. In a particular embodiment, X is O or CH₂. In a particular embodiment, the compound is selected from the group consisting of: adenosine, chemical name 2-(6-amino-9H-purin-9-yl)-5-(hydroxymethyl)tetrahydrofuran-3,4-diol; CPA, chemical name 2-(6-(cyclopentylamino)-9H-purin-9-yl)-5-(hydroxymethyl)tetrahydrofuran-3,4-diol; COPA, chemical name of 2-(2-chloro-6-(cyclopentylamino)-9H-purin-9-yl)-5-(hydroxymethyl)tetrahydrofuran-3,4-diol; 2′-MeCCPA, chemical name 2-(2-chloro-6-(cyclopentylamino)-9H-purin-9-yl)-5-(hydroxymethyl)-3-methyltetrahydrofuran-3,4-diol; Tecadenoson, chemical name 2-(hydroxymethyl)-5-(6-((tetrahydrofuran-3-yl)amino)-9H-purin-9-yl)tetrahydrofuran-3,4-diol; Selodenoson, chemical name 5-(6-(cyclopentylamino)-9H-purin-9-yl)-N-ethyl-3,4-dihydroxytetrahydrofuran-2-carboxamide; PJ-875, chemical name (5-(6-(cyclopentylamino)-9H-purin-9-yl)-3,4-dihydroxytetrahydrofuran-2-yl)methyl nitrate; ARA, chemical name 3-(2,2,2-trifluoroethyl)-5-(6-((1-(5-(trifluoromethyl)pyridin-2-yl)pyrrolidin-3-yl)amino)-9H-purin-9-yl)cyclopentane-1,2-diol; GR79236, chemical name 2-(6-((2-hydroxycyclopentyl)amino)-9H-purin-9-yl)tetrahydrofuran-3,4-diol; RPR-749; CVT-3619, chemical name 2-(((2-fluorophenyl)thio)methyl)-5-(6-((2-hydroxycyclopentyl)amino)-9H-purin-9-yl)tetrahydrofuran-3,4-diol; R-PIA, chemical name 2-(hydroxymethyl)-5-(6-((1-phenylpropan-2-yl)amino)-9H-purin-9-yl)tetrahydrofuran-3,4-diol; S-ENBA, chemical name 2-(6-(bicyclo[2.2.1]heptan-2-ylamino)-9H-purin-9-yl)-5-(hydroxymethyl)tetrahydrofuran-3,4-diol; AMP579, chemical name 5-(6-((1-(3-chlorothiophen-2-yl)butan-2-yl)amino)-9H-purin-9-yl)-N-ethyl-3,4-dihydroxytetrahydrofuran-2-carboxamide; GW493838, chemical name 2-(6-((2-hydroxycyclopentyl)amino)-9H-purin-9-yl)-5-(hydroxymethyl)tetrahydrofuran-3,4-diol; SDZ WAG 994, chemical name 2-(6-(cyclohexylamino)-9H-purin-9-yl)-5-(hydroxymethyl)-4-methoxytetrahydrofuran-3-ol; NNC 21-136, chemical name 2-(6-((1-(benzo[d]thiazol-2-ylthio)propan-2-yl)amino)-8-chloro-9H-purin-9-yl)-5-(hydroxymethyl)tetrahydrofuran-3,4-diol; 2-((2-fluorophenoxy)methyl)-5-(6-((tetrahydrofuran-3-yl)amino)-9H-purin-9-yl)tetrahydrofuran-3,4-diol; 2-(((2-fluorophenyl)thio)methyl)-5-(6-((tetrahydrofuran-3-yl)amino)-9H-purin-9-yl)tetrahydrofuran-3,4-diol; 3-(6-((3-chlorophenyl)(pyrrolidin-3-yl)amino)-9H-purin-9-yl)-5-(fluoromethyl)cyclopentane-1,2-diol; and 3-(6-(((3-chlorothiophen-2-yl)methyl)(ethyl)amino)-9H-purin-9-yl)-5-(fluoromethyl)cyclopentane-1,2-diol; optionally substituted with one or more, the same or different, substituents or salts thereof.

In a particular embodiment, the adenosine receptor agonist is a compound of formula II,

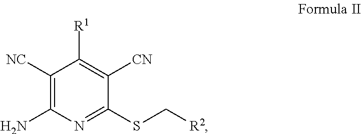

prodrugs, esters, and salts thereof wherein, R¹ and R² are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein each R¹ and R² are optionally substituted with one or more, the same or different, R⁸; R⁸ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁸ is optionally substituted with one or more, the same or different, R⁹; R⁹ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁹ is optionally substituted with one or more, the same or different, R¹⁰; and R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In a particular embodiment, R¹ is aryl optionally substituted with one or more, the same or different R⁸. In a particular embodiment, R² is alkyl or a heterocyclyl, and wherein R² is optionally substituted with one or more, the same or different R⁸.

In a particular embodiment, formula II has formula IIA,

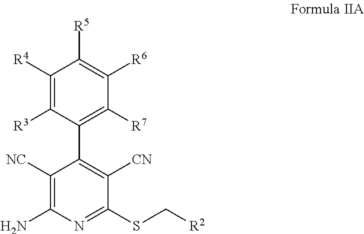

prodrugs, esters, or salts thereof wherein, R² is a heterocyclyl optionally substituted with one or more, the same or different, R⁸; R³, R⁴, R⁵, R⁶ and R⁷ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein each R³, R⁴, R⁵, R⁶ and R⁷ are optionally substituted with one or more, the same or different, R⁹; R⁸ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁸ is optionally substituted with one or more, the same or different, R⁹; R⁹ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁹ is optionally substituted with one or more, the same or different, R¹⁰; and R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In a particular embodiment, R⁵ is alkoxy optionally substituted with one or more, the same or different, R⁹. In a particular embodiment, the adenosine receptor agonist is capadenoson, chemical name 2-amino-6-(((2-(4-chlorophenyl)thiazol-4-yl)methyl)thio)-4-(4-(2-hydroxyethoxy)phenyl)pyridine-3,5-dicarbonitrile and 2-Amino-4-benzo[1,3]dioxol-5-yl-6-(2-hydroxyethylsulfanyl)pyridine-3,5-dicarbonitrile; optionally substituted with one or more, the same or different substituents or salts thereof.

In a particular embodiment, the adenosine receptor agonist is administered in combination with one or more additional antidepressants. In a particular embodiment, the one or more additional antidepressants is selected from the group consisting of: isocarboxazid, moclobemide, phenelzine, selegiline, tranylcypromine, citalopram, escitalopram, fluoxetine, fluvoxamine, paroxetine, sertraline, desvenlafaxine, duloxetine, milnacipran, venlafaxine, mianserin, mirtazapine, atomoxetine, mazindol, reboxetine, viloxazine, bupropion, tianeptine, agomelatine, amitriptyline, clomipramine, doxepin, imipramine, trimipramine, desipramine, nortriptyline and protriptyline.

In one embodiment, the disclosure is directed to a method of treating or preventing depression comprising administering an effective amount of an adenosine allosteric enhancer to a subject. In a particular embodiment, the subject is diagnosed with or is at risk of developing depression. In a particular embodiment, the adenosine allosteric enhancer is a 3-phenylthiophene derivative.

In a particular embodiment, the adenosine allosteric enhancer is a 8H-indeno[1,2-d]thiazole derivative. In a particular embodiment, the adenosine allosteric enhancer is a compound of formula III,

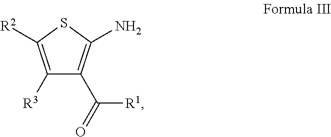

prodrugs, esters, or salts thereof wherein, R¹, R² and R³ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein each R¹, R² and R³ are optionally substituted with one or more, the same or different, R⁴; or R² and R³ form a carbocyclic or heterocyclic ring optionally substituted with one or more, the same or different, R⁴; R⁴ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁴ is optionally substituted with one or more, the same or different, R⁵; and R⁵ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In a particular embodiment, R¹ is alkoxy, benzyl, or aryl optionally substituted with one or more, the same or different, R⁴. In a particular embodiment, R² is hydrogen, halogen, alkyl or aryl. In a particular embodiment, R³ is aryl optionally substituted with one or more, the same or different, R⁴. In a particular embodiment, the compound of formula III is selected from the group consisting of: PD81,723, chemical name (2-amino-4,5-dimethylthiophen-3-yl)(3-(trifluoromethyl)phenyl)methanone; [2-amino-5-phenyl-4-(3-trifluoromethylphenyl)thiophen-3-yl]phenyl methanone; [2-amino-4-(3-(trifluoromethyl)phenyl)thiophen-3-yl]phenyl methanone; ethyl 2-amino-5-(4-chlorophenyl)-4-(3-trifluoromethylphenyl)thiophene-3-carboxylate; ethyl 2-amino-5-(4-methoxyphenyl)-4-(3-trifluoromethylphenyl)thiophene-3-carboxylate; benzyl 2-amino-4,5,6,7-tetrahydrobenzo[b]thiophene-3-carboxylate; benzyl 2-amino-5,6,7,8-tetrahydrocyclohepta[b]thiophene-3-carboxylate; benzyl 2-amino-4,5,6,7-tetrahydrobenzo[b]thiophene-3-carboxamide; and benzyl 2-amino-5,6,7,8-tetrahydrocyclohepta[b]thiophene-3-carboxamide; optionally substituted with one or more, the same or different, substituents or salts thereof.

In a particular embodiment, the adenosine allosteric enhancer is a compound of formula IV,

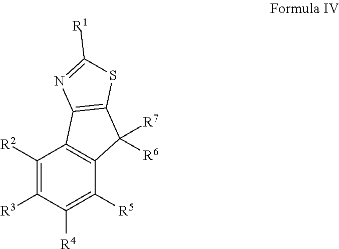

prodrugs, ester, or salts thereof wherein, R¹, R², R³, R⁴, R⁵, R⁶ and R⁷ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein each R¹, R², R³, R⁴, R⁵, R⁶ and R⁷ are optionally substituted with one or more, the same or different, R⁸; R⁸ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁸ is optionally substituted with one or more, the same or different, R⁹; and R⁹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In a particular embodiment, R¹ is amino. In a particular embodiment, R⁵ is hydroxy substituted with alkanoyl optionally substituted with one or more, the same or different R⁹. In a particular embodiment, the adenosine allosteric enhancer is administered in combination with an adenosine receptor agonist. In a particular embodiment, the adenosine allosteric enhancer is administered in combination with one or more additional antidepressants.

In one embodiment, the disclosure is directed to a pharmaceutical composition comprising an adenosine receptor agonist and one or more additional antidepressants. In a particular embodiment, the adenosine receptor agonist is selected from the group consisting of: adenosine, chemical name 2-(6-amino-9H-purin-9-yl)-5-(hydroxymethyl)tetrahydrofuran-3,4-diol; CPA, chemical name 2-(6-(cyclopentylamino)-9H-purin-9-yl)-5-(hydroxymethyl)tetrahydrofuran-3,4-diol; CCPA, chemical name of 2-(2-chloro-6-(cyclopentylamino)-9H-purin-9-yl)-5-(hydroxymethyl)tetrahydrofuran-3,4-diol; 2′-MeCCPA, chemical name 2-(2-chloro-6-(cyclopentylamino)-9H-purin-9-yl)-5-(hydroxymethyl)-3-methyltetrahydrofuran-3,4-diol; Tecadenoson, chemical name 2-(hydroxymethyl)-5-(6-((tetrahydrofuran-3-yl)amino)-9H-purin-9-yl)tetrahydrofuran-3,4-diol; Selodenoson, chemical name 5-(6-(cyclopentylamino)-9H-purin-9-yl)-N-ethyl-3,4-dihydroxytetrahydrofuran-2-carboxamide; PJ-875, chemical name (5-(6-(cyclopentylamino)-9H-purin-9-yl)-3,4-dihydroxytetrahydrofuran-2-yl)methyl nitrate; ARA, chemical name 3-(2,2,2-trifluoroethyl)-5-(6-((1-(5-(trifluoromethyl)pyridin-2-yl)pyrrolidin-3-yl)amino)-9H-purin-9-yl)cyclopentane-1,2-diol; GR79236, chemical name 2-(6-((2-hydroxycyclopentyl)amino)-9H-purin-9-yl)tetrahydrofuran-3,4-diol; RPR-749; CVT-3619, chemical name 2-(((2-fluorophenyl)thio)methyl)-5-(6-((2-hydroxycyclopentyl)amino)-9H-purin-9-yl)tetrahydrofuran-3,4-diol; R-PIA, chemical name 2-(hydroxymethyl)-5-(6-((1-phenylpropan-2-yl)amino)-9H-purin-9-yl)tetrahydrofuran-3,4-diol; S-ENBA, chemical name 2-(6-(bicyclo[2.2.1]heptan-2-ylamino)-9H-purin-9-yl)-5-(hydroxymethyl)tetrahydrofuran-3,4-diol; AMP579, chemical name 5-(6-((1-(3-chlorothiophen-2-yl)butan-2-yl)amino)-9H-purin-9-yl)-N-ethyl-3,4-dihydroxytetrahydrofuran-2-carboxamide; GW493838, chemical name 2-(6-((2-hydroxycyclopentyl)amino)-9H-purin-9-yl)-5-(hydroxymethyl)tetrahydrofuran-3,4-diol; SDZ WAG 994, chemical name 2-(6-(cyclohexylamino)-9H-purin-9-yl)-5-(hydroxymethyl)-4-methoxytetrahydrofuran-3-ol; NNC 21-136, chemical name 2-(6-((1-(benzo[d]thiazol-2-ylthio)propan-2-yl)amino)-8-chloro-9H-purin-9-yl)-5-(hydroxymethyl)tetrahydrofuran-3,4-diol; 2-((2-fluorophenoxy)methyl)-5-(6-((tetrahydrofuran-3-yl)amino)-9H-purin-9-yl)tetrahydrofuran-3,4-diol; 2-(((2-fluorophenyl)thio)methyl)-5-(6-((tetrahydrofuran-3-yl)amino)-9H-purin-9-yl)tetrahydrofuran-3,4-diol; 3-(6-((3-chlorophenyl)(pyrrolidin-3-yl)amino)-9H-purin-9-yl)-5-(fluoromethyl)cyclopentane-1,2-diol; and 3-(6-(((3-chlorothiophen-2-yl)methyl)(ethyl)amino)-9H-purin-9-yl)-5-(fluoromethyl)cyclopentane-1,2-diol; Capadenoson, chemical name 2-amino-6-(((2-(4-chlorophenyl)thiazol-4-yl)methyl)thio)-4-(4-(2-hydroxyethoxy)phenyl)pyridine-3,5-dicarbonitrile and 2-Amino-4-benzo[1,3]dioxol-5-yl-6-(2-hydroxyethylsulfanyl)pyridine-3,5-dicarbonitrile; and derivatives and salts thereof. In a particular embodiment, the one or more additional antidepressants is selected from the group consisting of: isocarboxazid, moclobemide, phenelzine, selegiline, tranylcypromine, citalopram, escitalopram, fluoxetine, fluvoxamine, paroxetine, sertraline, desvenlafaxine, duloxetine, milnacipran, venlafaxine, mianserin, mirtazapine, atomoxetine, mazindol, reboxetine, viloxazine, bupropion, tianeptine, agomelatine, amitriptyline, clomipramine, doxepin, imipramine, trimipramine, desipramine, nortriptyline and protriptyline.

In one embodiment, the disclosure is directed to the use of any of the compounds identified or described herein for the production of a medicament for the treatment or prevention of a disease, disorder or condition is selected from the group consisting of: depression or depressive-like symptoms, sleep disorders in the elderly, Parkinson's disease, Alzheimer's disease, epilepsy, schizophrenia and symptoms experienced by recovering alcoholics.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-F describe results of sleep deprivation tests in mouse models. Mice expressing a dominant negative SNARE protein selectively in astrocytes do not demonstrate the beneficial effects of sleep deprivation on depression or depressive-like symptoms. FIG. 1A is a schematic diagram of tTA and tetO driven expression of GFP and dnSNARE, which can be repressed by doxycycline. FIG. 1B shows confocal images of GFP expression in the frontal cortex of dnSNARE mice both off (left) and on (right) doxycycline. Quantification of average GFP intensity in frontal cortex (FIG. 10) and hippocampal dentate gyrus (FIG. 1D) of dnSNARE and WT littermate control mice both on and off doxycycline. FIG. 1E are representative heat maps of WT, WT sleep deprived, dnSNARE, and dnSNARE sleep deprived animal performance in the last four minutes of the forced swim task. Colors in the warm spectrum represent increased time spent, and thus increased immobility. FIG. 1F are graphs showing the quantification of time spent immobile in the last four minutes of the forced swim task in dnSNARE and WT control mice, with or without sleep deprivation treatment. In comparison to WT controls, dnSNARE mice do not show a reduction in time immobile following sleep deprivation. In contrast, dnSNARE mice do show a reduction in time spent immobile, similar to that of WT controls, when treated with imipramine, quantified in FIG. 1G. Similar to the forced swim test showing the beneficial effects of sleep deprivation on immobile time, FIG. 1H are graphs for WT mice using the tail suspension test, whereas dnSNARE mice again do not demonstrate a reduction in immobile time with sleep deprivation.

FIGS. 2A-D show results of interference with adenosine signaling using A1R knockout mice or pharmacological blockade with intracerebroventricular cyclopentyltheophylline. Lack of A1R signaling blocks the beneficial effects of sleep deprivation on depressive-like symptoms. FIG. 2A is a series of light microscope images of A1R immunohistochemistry for A1R^{+/+} and A1R^{-/-} brain. FIG. 2B is a series of heat maps of representative performance of A1R^{+/+}, A1R^{-/-} and i.c.v. CPT-treated mice during the last four minutes of the forced swim test. Quantification of time immobile shows that knockout of A1R, or blocking A1R signaling using i.c.v. CPT, prevents the beneficial effects of sleep deprivation on depressive-like symptoms in both the forced swim test (FIG. 2C) and the tail suspension test (FIG. 2D).

FIGS. 3A and 3B are plots showing short term but not extended total sleep deprivation maintains wakefulness dependent inhibition by astrocyte derived adenosine. FIG. 3A shows characteristic field excitatory postsynaptic potential (fEPSP) measured in at the Schaeffer collateral synapse of hippocampal area CA1 before (black) or following (Red) application of 8-cyclopentyltheophylline (CPT, 200 nM). FIG. 3B shows the increase in slope of the fEPSP following application CPT (percentage of baseline) provides a relative measure of extracellular adenosine (Adenosine tone). Adenosine tone increases following wakefulness and declines during sleep in slices from WT but not dnSNARE or AdorA1 Knockout (A1KO) mice. Transient total sleep deprivation (12 hours) maintains the wakefulness dependent increase in adenosine tone but this increase is lost following extended sleep deprivation (72 hours). Significant effect of genotype p<0.001.

FIGS. 4A-D are results showing the adenosine receptor agonist 2-chloro-N(6)-cyclopentyladenosine (CCPA) alters network patterns of electrical activity and sleep architecture. FIG. 4A: The percent time awake is decreased during CCPA administration (arrows). FIG. 4B: Normalized power spectrum at ZT=12 during wakefulness at hours 12, 36, 84 and 108 after initial drug administration. Data are normalized to the total power of the EEG between 0.5 and 40 Hz. FIG. 4C: Normalized power spectrum from 0.5 to 4.0 Hz for wake states at 12, 36, 84 and 108 hours after initial drug administration. FIG. 4D: Normalized power during wake of the delta (0.5-4 Hz), theta (5-8 Hz) alpha (9-13 Hz) and gamma (20-40 Hz). *: p≦0.05, **: p≦0.01, ***: p≦0.001.

FIGS. 5A-C are data showing treatment of mice with the adenosine receptor agonist, 2-chloro-N(6)-cyclopentyladenosine (CCPA), partially mimics the beneficial effects of sleep deprivation on depressive-like symptoms. Activation of A1R with CCPA leads to sustained antidepressive-like behaviors. FIG. 5A: representative heatmaps showing dwell time during the last four minutes of the forced swim test following COPA treatment and sleep deprivation (12 h). FIG. 5B: CCPA significantly reduces immobility in the forced swim test, an effect that is sustained for 36 h following onset of CCPA administration. FIG. 5C: validation of forced swim test results using an additional test of depressive-like symptoms, the sucrose consumption test. *p value, *: p≦0.05, **: p≦0.01, ***: p≦0.001.

FIGS. 6A-D show the apparatus used for and data obtained from tail suspension tests. This demonstrates that sleep deprivation is successfully modeled in mice. FIG. 6A: the Pinnacle Technologies (2721 Oregon St., Lawrence, Kans. 66046) apparatus for automated sleep deprivation in mice. FIG. 6B: sleep deprivation affects vigilance states in mice by only allowing minor transitions from wake to NREM and no REM sleep. FIG. 6C: the random setting on the machine produced only minor transitions to NREM from wake. FIG. 6D: the duration of sleep deprivation is critical for the therapeutic benefits seen the forced swim task, with 12 hour but not 72 hours of sleep deprivation being effective. *: p≦0.05, **: p≦0.01, ***: p≦0.001.

FIGS. 7A-C are plots showing the effects of various treatments and mouse strains on open field behavior. FIG. 7A: Wildtype, dnSNARE, and A1R^{-/-} mice and wildtype mice treated with sleep deprivation, CPT or CCPA, do not differ from each other in total distance traveled in the open field test demonstrating that these genotypes and treatments do not cause a generalized stimulation of mouse behavior. Importantly genetic and pharmacological manipulations do not change locomotor activity in the open field test, allowing one to conclude that changes in immobility seen in forced swim and tail suspension tests are related to antidepressive effects of sleep deprivation and pharmacological manipulations. FIG. 7B: vehicle and CCPA treated mice do not differ in the latency to fall from the rotorod test suggesting the treatment does not cause motor gross changes in motor behavior. FIG. 7C: animals show increased percent wake at 12 h after administration of the adenosine receptor agonist, CCPA, but this effect is no longer present at 36 h following CCPA.

FIGS. 8A-C are plots showing the adenosine receptor agonist CCPA alters network patterns of electrical activity and sleep architecture. FIG. 8A: ratio (CCPA/Vehicle) of normalized power spectrum at ZT=12 during wakefulness at hours 12, 36, 84 and 108 after initial drug administration. FIG. 8B: ratio (CCPA/Vehicle) of normalized power spectrum from 0.5 to 4.5 Hz for wake states at hours 12, 36, 84 and 108 after initial drug administration. FIG. 8C: ratio (CCPA/Vehicle) of normalized power during wake of the delta (0.5-4 Hz), theta (5-8 Hz) alpha (9-13 Hz) and gamma (20-40 Hz). *: p≦0.05, **: p≦0.01, ***: p≦0.001.

FIG. 9 shows results indicating the use adenosine biosensors to measure the accumulation of adenosine in situ (frontal cortical brain slice) and in vivo.

FIG. 10 shows data using the peptide ligand TFLLR (SEQ ID NO:1), which activates PAR1, an astrocyte-dependent elevation of extracellular adenosine that is attenuated in dnSNARE slices is shown

### DETAILED DESCRIPTION

Sleep abnormalities are co-morbid with many psychiatric conditions including major depressive disorder (MDD); a total night of sleep deprivation, however, has dramatic and immediate antidepressive actions in the clinical population (Germain, A. & Kupfer, D., Hum. Psychopharmacol., 23:571-585, 2008; Giedke, H. & Schwarzler, F., Sleep Med. Rev., 6:361-77, 2002; Gillin, J., Prog. Neuropsychopharmacol. Biol. Psychiatry, 7:351-64, 1983; Landsness, E. et al., J. Psychiatr. Res., 45:1019-26, 2011; Le Bon, O., Dialogues Clin. Neurosci., 7:305-13, 2005; Monteleone, P. et al., Prog. Neuropsychopharmacol. Biol. Psychiatry, 35:1569-74, 2011; Pflug, B. & Tolle, R., Nervenarzt., 42:117-124, 1971; Svestka, J., Neuro. Endocrinol. Lett., 29(Suppl 1):65-92, 2008; Wirz-Justice, A. & van den Hoofdakker, R., Biol. Psychiatry, 46:445-53, 1999; Wu, J. et al., Biol. Psychiatry, 66:298-301, 2009). Selective slow-wave sleep deprivation is antidepressive and fMRI shows that sleep deprivation leads to amplified reward-relevant reactivations in mesolimbic reward pathways (Gujar, N. et al., J. Neurosci., 31:4466-74, 2011). The mechanism underlying these antidepressive effects of sleep deprivation, however, had previously been unknown.

Current pharmacological treatments for MDD take weeks for clinical efficacy, limiting the ability to bring instant relief to suicidal patients. In contrast, a non-pharmacological intervention that rapidly alleviates symptoms of depression is a night of total sleep deprivation (Germain, A. et al., Sleep Med. Rev., 12:185-95, 2008) which is effective in approximately 60% of depressed patients (Hemmeter, U. et al., Biol. Psychiatry, 43:829-39, 1998; Hemmeter, U. et al., Expert Rev. Neurother., 10:1101-15, 2010).

A close relationship exists between MDD and sleep disorders. Symptoms of MDD involve altered sleep homeostasis that can be measured by changes in slow wave activity (SWA) (Goldstein, M. et al., Acta. Psychiatr. Scand., 125:468-77, 2012). Pronounced changes in both sleep bout duration and the EEG spectrum have been shown to be hallmark changes in depressed patients. The reduction in depressive symptoms observed following sleep deprivation correlates with overnight dissipation of fronto-central SWA on baseline sleep, and with the rebound in right frontal all-night SWA during recovery sleep (Landsness, E. et al., Clin. Neurophysiol., 122:2418-25, 2011). Selective slow wave sleep deprivation promotes antidepressant actions in patients, although the mechanism was unknown. Evidence suggested that the amplitude and slope of slow waves is related to the number, strength and efficacy of the synaptic connections within the network (Hanlon, E. et al., Curr. Top. Med. Chem., 11:2472-82, 2011). The idea of regulating synaptic strength in rapid therapies for depression is pertinent, given that another rapidly acting antidepressant, electroconvulsive therapy (ECT), may act via increasing synaptic strength in prefrontal cortex (PFC) (Duman, R. et al., J. Ect., 14:181-93, 1998; Layergne, F. & Jay, T., Front. Neurosci., 4:192, 2010). In addition, optogenetic stimulation of the PFC, resulting in increased synaptic strength, exerts antidepressant effect in animal models (Covington, H. et al., J. Neurosci., 30:16082-90, 2010).

Because the effects of sleep deprivation on depression are not long lasting, sleep deprivation is not always used clinically. If the mechanism mediating this action were identified, however, it would be possible to develop therapeutics that target this pathway as a new treatment for certain forms of depression and depressive disorders. Although it is difficult to model all aspects of depression in mice, it is known that stimuli such as sleep deprivation and electroconvulsive therapy that are clinically effective, lead to robust changes in depressive-like behaviors in mice. Described herein are the therapeutic targets, methods of identifying therapeutic targets, therapeutic agents, and methods of using therapeutics agents that are based on the discovery of the mechanism that leads to alleviation of depression and depressive-like symptoms due to sleep deprivation. The identification of the mechanism is validated using a sucrose consumption test that models of a different aspect of depression, anhedonia.

Astrocytes regulate responses to sleep deprivation. Described herein are data that elucidate the role of this glial pathway in mediating antidepressive-like actions of sleep deprivation in C57Bl/6J mice. Twelve hours of sleep deprivation produces a robust reduction of depressive-like behaviors that requires astrocytic signaling to adenosine (A1) receptors. Moreover, sleep deprivation activates synaptic A1 receptor pathway(s) and pharmacological activation of A1R, using central administration of the agonist CCPA (2-chloro-N(6)-cyclopentyladenosine), independent of sleep deprivation promotes antidepressant-like effects. These results point towards a novel strategy to bring instant relief to depressive symptoms through the activation of wakefulness-dependent glial pathways that activate A1R signaling systems. Adenosine signaling has also been linked to depression, however controversy exists whether adenosine (and its agonists) act in an antidepressant (Kaster, M. et al., Neurosci. Lett., 355:21-24, 2004) or a depressant (El Yacoubi, M. et al., Neurology, 61(Suppl):S82-S87, 2003) manner. Because SNARE-sensitive astrocyte-mediated signaling contributes to the immediate effects of sleep deprivation on slow-wave activity, as well as compensatory sleep time, the present disclosure addresses whether this signaling pathway contributes to antidepressive-like actions of sleep deprivation in murine behavioral despair models of depression.

As used herein, “adenosine receptor” refers to all subtypes of adenosine receptors. Compounds and methods disclosed herein can refer to adenosine receptors or to specific subtypes, e.g., the adenosine A1 receptor.

Additional A1R agonists can be structurally similar to adenosine (“adenosine analogs”). The following described the structure of adenosine and provides guidance as to how such analogs can be made:

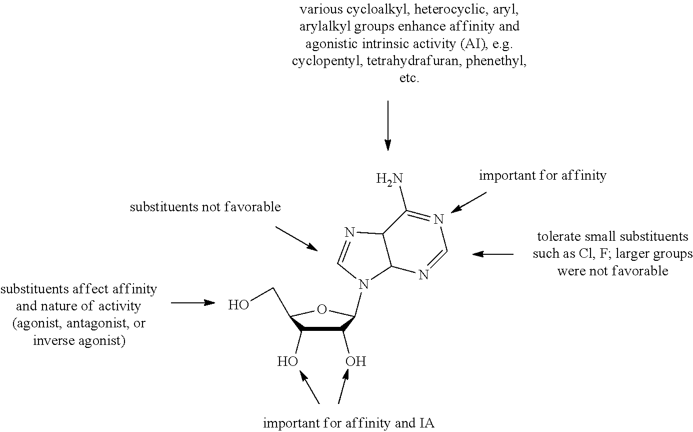

Traditional therapeutics for depression are based on monoamine targets to modulate mood. These strategies are limited by the delayed onset of beneficial effects, which can take several days to be achieved. In contrast, the adenosine signaling pathway activated by sleep deprivation is rapidly beneficial and represents a novel strategy to bring relief to suicidal patients where a delay in therapeutic benefits could be devastating. The identification of A1R agonists, e.g., CCPA, as a novel, fast acting compound, represents an improvement on sleep deprivation as a therapeutic in the treatment of depression. Other compounds known to activate the A1R signaling pathway specifically include, for example, N6-cyclopentyladenosine, 2′-MeCCPA, N-[(1S,2S)-2-Hydroxycyclopentyl]adenosine (commercially available as GR 79236), and N-Cyclohexyl-2′-O-methyladenosine (commercially available as SDZ WAG 994). Although the results described herein refer to the use of A1R-specific agonists, other agonists that activate other classes of adenosine receptors in addition to A1R can also be used. It is understood by one of skill in the art that use of non-specific agonist can lead to effects, either deleterious or beneficial, other than treatment of depression or depressive-like symptoms. The use of non-specific agonist to activate, for example, signaling through the adenosine 2A receptor (A2AR), adenosine 2B receptor (A2BR), and/or adenosine A3 receptor (A3R) singly or in any combination with A1R signaling, is encompassed within the scope of the present disclosure.

The results described herein indicate the use of A1R agonists for treatment of depression and depressive-like symptoms. The present disclosure, therefore, relates to methods for treating depression or depressive-like symptoms by administering an effective amount of an A1R agonist or downstream effector molecule that activates the A1R signaling pathway. As used herein, “agonist” refers to an effector molecule that activates the activity of another molecule, e.g., the A1R. An agonist can be, for example, a small molecule, e.g., CCPA, an analog of another agonist, an antagonist of an antagonist, or analogs or derivatives thereof. An agonist can be, for example, an activating enzyme or precursor to an agonist (e.g., a downstream effector molecule that, for example, leads to increased levels of adenosine, thereby activating A1R). An agonist can be, for example, an activating antibody or an inhibitory antibody of, for example, an A1R antagonist. Examples of A1R agonists include, but are not limited to, the following:

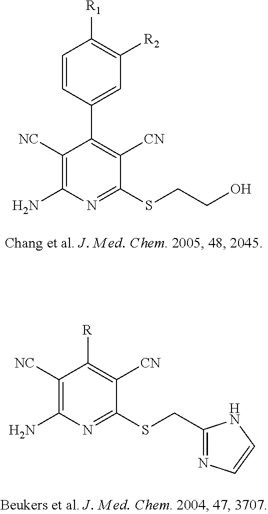

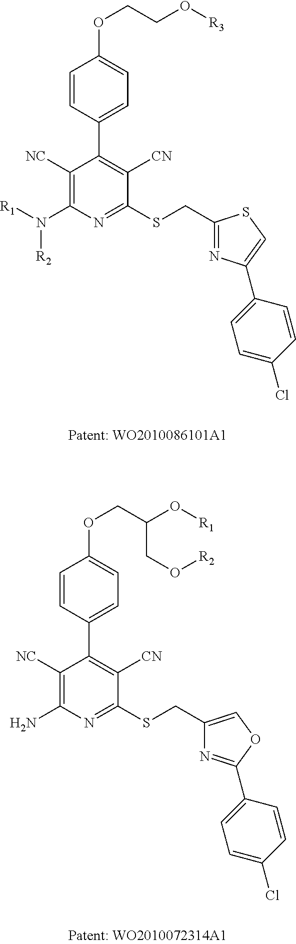

Small molecule agonists can be, for example, structurally related to the natural A1R activating effector molecule, adenosine. Small molecule agonists can be structurally related to other A1R agonists. Small molecule A1R agonists are known, but have previously not been considered for their use in treating depression or depressive-like symptoms. Use of an A1R agonist for treating depression or depressive-like symptoms is supported by the findings described herein that treatment, while not wishing to be bound by theory, is dependent on the activation of the A1R signaling pathway, and, therefore, for the purposes of the present disclosure, any such effector molecule that activates A1R signaling is envisioned for use in treating depression or depressive-like symptoms.

In addition, allosteric effector (“enhancer”) molecules can be used to activate A1R signaling, for example, by enhancing the effects of endogenous adenosine. Examples of such allosteric effectors include, but are not limited to, the following:

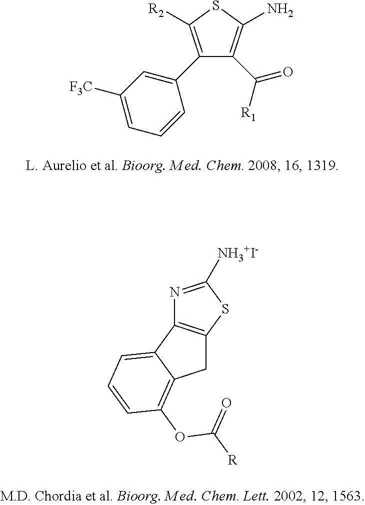

One embodiment of the disclosure is directed to using one or more of the identified agents identified herein or identified through the use of a screen described herein to treat depression or depressive-like symptoms. The compounds identified herein or identified through the screens described herein can be delivered in a variety of formulations and amounts to achieve desired effects.

“Treatment” refers to the administration of medicine or the performance of medical procedures with respect to a patient or subject, for either prophylaxis (prevention) or to cure or reduce the symptoms of the infirmity or malady in the instance where the patient is afflicted. Prevention of depression or depressive-like symptoms is included within the scope of treatment. The compounds described herein or identified through methods described herein can be used as part of a treatment regimen in therapeutically effective amounts. A “therapeutically effective amount” is an amount sufficient to decrease, prevent or ameliorate the symptoms associated with a medical condition. e.g., depression or depressive-like symptoms. The present disclosure, for example, is directed to treatment using a therapeutically effective amount of a compound sufficient to treat depression or depressive-like symptoms.

The terms “patient” and “subject” mean all mammals including humans.

The treatment(s) described herein are understood to utilize formulations including compounds identified herein or identified through methods described herein and, for example, salts, solvates and co-crystals of the compound(s). The compounds of the present disclosure can exist in unsolvated as well as solvated forms with pharmaceutically acceptable solvents such as, for example, water, ethanol, and the like. In general, the solvated forms are considered equivalent to the unsolvated forms for the purposes of the present disclosure.

The term “pharmaceutically acceptable salts, esters, amides and prodrugs” as used herein refers to those carboxylate salts, amino acid addition salts, esters, amides, prodrugs and inclusion complexes of the compounds of the present disclosure that are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

The term “prodrug” refers to compounds that are rapidly transformed in vivo to yield the parent compounds of the above formula, for example, by hydrolysis in blood (T. Higuchi and V. Stella, “Pro-drugs as Novel Delivery Systems,” Vol. 14 of the A.C.S. Symposium Series; Bioreversible Carriers in Drug Design, ed. Edward B. Roche, American Pharmaceutical Association and Pergamon Press, 1987; both of which are incorporated herein by reference in their entirety). Activation in vivo may come about by chemical action or through the intermediacy of enzymes. Microflora in the GI tract may also contribute to activation in vivo.

The term “solvate” refers to a compound in the solid state, wherein molecules of a suitable solvent are incorporated. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Co-crystals are combinations of two or more distinct molecules arranged to create a unique crystal form whose physical properties are different from those of its pure constituents (Remenar, J. et al., 2003. J. Am. Chem. Soc., 125:8456-8457) and fluoxetine. Inclusion complexes are described in Remington: The Science and Practice of Pharmacy 19.sup.th Ed. (1995) volume 1, page 176-177. The most commonly employed inclusion complexes are those with cyclodextrins, and all cyclodextrin complexes, natural and synthetic, with or without added additives and polymer(s), as described in U.S. Pat. Nos. 5,324,718 and 5,472,954. The disclosures of Remenar, Remington and the '718 and '954 patents are incorporated herein by reference in their entireties.

The compounds can be presented as salts. The term “pharmaceutically acceptable salt” refers to salts whose counter ion derives from pharmaceutically acceptable non-toxic acids and bases. Suitable pharmaceutically acceptable base addition salts for the compounds of the present disclosure include, but are not limited to, metallic salts made from aluminum, calcium, lithium, magnesium, potassium, sodium and zinc or organic salts made from lysine, N,N-dialkyl amino acid derivatives (e.g., N,N-dimethylglycine, piperidine-1-acetic acid and morpholine-4-acetic acid), N,N′-dibenzylethylenediamine, chloroprocaine, choline, diethanolamine, ethylenediamine, meglumine (N-methylglucamine) and procaine. Where the compounds contain a basic residue, suitable pharmaceutically acceptable base addition salts for the compounds include, for example, inorganic acids and organic acids. Examples include acetate, benzenesulfonate (besylate), benzoate, bicarbonate, bisulfate, carbonate, camphorsulfonate, citrate, ethanesulfonate, fumarate, gluconate, glutamate, bromide, chloride, isethionate, lactate, maleate, malate, mandelate, methanesulfonate, mucate, nitrate, pamoate, pantothenate, phosphate, succinate, sulfate, tartrate, p-toluenesulfonate, and the like (Barge, S et al., 1977. J. Pharm. Sci., 66:1-19, the entire contents of which are incorporated herein by reference).

Diluents that are suitable for use in the pharmaceutical composition of the present disclosure include, for example, pharmaceutically acceptable inert fillers such as microcrystalline cellulose, lactose, sucrose, fructose, glucose dextrose, or other sugars, dibasic calcium phosphate, calcium sulfate, cellulose, ethylcellulose, cellulose derivatives, kaolin, mannitol, lactitol, maltitol, xylitol, sorbitol, or other sugar alcohols, dry starch, saccharides, dextrin, maltodextrin or other polysaccharides, inositol or mixtures thereof. The diluent can be, for example, a water-soluble diluent. Examples of preferred diluents include, for example: microcrystalline cellulose such as Avicel PH112, Avicel PH101 and Avicel PH102 available from FMC Corporation; lactose such as lactose monohydrate, lactose anhydrous, and Pharmatose DCL 21; dibasic calcium phosphate such as Emcompress; mannitol; starch; sorbitol; sucrose; and glucose. Diluents are carefully selected to match the specific composition with attention paid to the compression properties. The diluent can be used in an amount of about 2% to about 80% by weight, about 20% to about 50% by weight, or about 25% by weight of the treatment formulation.

Other agents that can be used in the treatment formulation include, for example, a surfactant, dissolution agent and/or other solubilizing material. Surfactants that are suitable for use in the pharmaceutical composition of the present disclosure include, for example, sodium lauryl sulphate, polyethylene stearates, polyethylene sorbitan fatty acid esters, polyoxyethylene castor oil derivatives, polyoxyethylene alkyl ethers, benzyl benzoate, cetrimide, cetyl alcohol, docusate sodium, glyceryl monooleate, glyceryl monostearate, glyceryl palmitostearate, lecithin, medium chain triglycerides, monoethanolamine, oleic acid, poloxamers, polyvinyl alcohol and sorbitan fatty acid esters. Dissolution agents increase the dissolution rate of the active agent and function by increasing the solubility of the active agent. Suitable dissolution agents include, for example, organic acids such as citric acid, fumaric acid, tartaric acid, succinic acid, ascorbic acid, acetic acid, malic acid, glutaric acid and adipic acid, which may be used alone or in combination. These agents can also be combined with salts of the acids, e.g., sodium citrate with citric acid, to produce a buffer system. Other agents that can be used to alter the pH of the microenvironment on dissolution include salts of inorganic acids and magnesium hydroxide.

Disintegrants that are suitable for use in the pharmaceutical composition of the present disclosure include, for example, starches, sodium starch glycolate, crospovidone, croscarmellose, microcrystalline cellulose, low substituted hydroxypropyl cellulose, pectins, potassium methacrylate-divinylbenzene copolymer, poly(vinyl alcohol), thylamide, sodium bicarbonate, sodium carbonate, starch derivatives, dextrin, beta cyclodextrin, dextrin derivatives, magnesium oxide, clays, bentonite and mixtures thereof.

The active ingredient of the present disclosure can be mixed with excipients, which are pharmaceutically acceptable and compatible with the active ingredient and in amounts suitable for use in the therapeutic methods described herein. Various excipients can be homogeneously mixed with the active agent of the present disclosure as would be known to those skilled in the art. The active agent, for example, can be mixed or combined with excipients such as but not limited to microcrystalline cellulose, colloidal silicon dioxide, lactose, starch, sorbitol, cyclodextrin and combinations of these.

Compositions of the present disclosure can also optionally include other therapeutic ingredients, anti-caking agents, preservatives, sweetening agents, colorants, flavors, desiccants, plasticizers, dyes, and the like.

In certain embodiments, the compositions are administered in combination with a second antidepressant (antidepressant agent).

Any such optional ingredient must, of course, be compatible with the compound of the disclosure to insure the stability of the formulation. The dose range for adult humans is generally from 0.1 μg to 10 g/day orally. Tablets or other forms of presentation provided in discrete units can conveniently contain an amount of compound of the disclosure that is effective at such dosage or as a multiple of the same, for instance, units containing 0.1 mg to 500 mg, usually around 5 mg to 200 mg. The precise amount of compound administered to a patient will be the responsibility of the attendant physician. The dose employed will depend on a number of factors, including, for example, the age and sex of the patient, the precise disorder being treated, and its severity. The frequency of administration depends on the pharmacodynamics of the individual compound and the formulation of the dosage form, which is optimized by methods known in the art (e.g., controlled or extended release tablets, enteric coating etc.).

In certain embodiments, the compounds disclosed herein are optionally substituted with one or more substituents. The term “substituted” refers to a molecule wherein at least one hydrogen atom is replaced with a substituent. When substituted, one or more of the groups are “substituents.” The molecule can be multiply substituted. In the case of an oxo substituent (“═O”), two hydrogen atoms are replaced. Example substituents within this context include, for example, halogen, hydroxy, alkyl, alkoxy, alkanoyl, nitro, cyano, oxo, carbocyclyl, carbocycloalkyl, heterocarbocyclyl, heterocarbocycloalkyl, aryl, arylalkyl, heterocarbocyclyl, heteroaryl, heteroarylalkyl, —NRaRb, —NRaC(═O)Rb, —NRaC(═O)NRaNRb, —NRaC(═O)ORb, —NRaSO2Rb, —C(═O)Ra, —C(═O)ORa, —C(═O)NRaRb, —OC(═O)NRaRb, —ORa, —SRa, —SORa, —S(═O)2Ra, —OS(═O)2Ra and —S(═O)2ORa. Ra and Rb in this context may be the same or different and independently hydrogen, halogen, hydroxyl, alkyl, alkoxy, alkanoyl, amino, alkylamino, dialkylamino, alkylthiol, carbocyclyl, carbocycloalkyl, heterocarbocyclyl, heterocarbocycloalkyl, aryl, arylalkyl, heteroaryl, heteroarylalkyl. The term “optionally substituted,” as used herein, means that substitution is optional and therefore it is possible for the designated atom or compound is unsubstituted.

As used herein, “alkyl” means a noncyclic straight chain or branched, unsaturated or saturated hydrocarbon such as those containing from 1 to 10 carbon atoms, while the term “lower alkyl” or “C₁₋₆ alkyl” has the same meaning as alkyl but contains from 1 to 6 carbon atoms. The term “higher alkyl” has the same meaning as alkyl but contains from 7 to 10 carbon atoms. Representative saturated straight chain alkyls include, for example, methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl,” respectively). Representative straight chain and branched alkenyls include, for example, ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include, for example, cyclopropyl, cyclobutyl, cyclopentyl, cyclohexyl, and the like; while unsaturated carbocycles include, for example, cyclopentenyl and cyclohexenyl, aryls and the like.

“Heterocarbocycles” or “heterocarbocyclyl” groups are carbocycles that contain from one to four heteroatoms independently selected from, for example, nitrogen, oxygen and sulfur (which may be saturated or unsaturated (but not aromatic)), monocyclic or polycyclic, and wherein the nitrogen and sulfur heteroatoms can be optionally oxidized, and the nitrogen heteroatom can be optionally quaternized. Heterocarbocycles include, for example, morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl.

As used herein, “heteroaryl” refers an aromatic heterocarbocycle having one to four heteroatoms selected from, for example, nitrogen, oxygen and sulfur, and containing at least one carbon atom, including both mono- and polycyclic ring systems. Polycyclic ring systems can, but are not required to, contain one or more non-aromatic rings, as long as one of the rings is aromatic. Representative heteroaryls are, for example, furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes, for example, N-alkylated derivatives such as a 1-methylimidazol-5-yl substituent.

As used herein, “heterocycle” or “heterocyclyl” refers to mono- and polycyclic ring systems having one to four heteroatoms selected from, for example, nitrogen, oxygen and sulfur, and containing at least one carbon atom. The mono- and polycyclic ring systems can be aromatic, non-aromatic or mixtures of aromatic and non-aromatic rings. Heterocycle includes heterocarbocycles, heteroaryls, and the like.

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy.

“Alkylamino” refers an alkyl group as defined above with the indicated number of carbon atoms attached through an amino bridge. An example of an alkylamino is methylamino, (e.g., —NH—CH₃).

“Alkanoyl” refers to an alkyl as defined above with the indicated number of carbon atoms attached through a carbonyl bridge (i.e., —(C═O)alkyl).

The compounds of this disclosure can exist in radiolabeled form, i.e., the compounds can contain one or more atoms containing an atomic mass or mass number different from the atomic mass or mass number most commonly found in nature. Radioisotopes of, for example, hydrogen, carbon, phosphorous, fluorine, and chlorine include ²H, ³H, ¹³C, ¹⁴C, ¹⁵N, ³⁵S, ¹⁸F and ³⁶Cl, respectively. Compounds that contain those radioisotopes and/or other radioisotopes of other atoms are within the scope of this disclosure. Radiolabeled compounds of the present disclosure and prodrugs thereof can generally be prepared by methods well known to those skilled in the art.

The compounds described herein can contain asymmetric centers and can thus give rise to enantiomers, diastereomers and other stereoisomeric forms. Each chiral center can be defined in terms of absolute stereochemistry as (R)- or (S)-. The present disclosure is meant to include all such possible isomers, as well as, their racemic and optically pure forms. Optically active (R)- and (S)-isomers can be prepared using chiral synthons or chiral reagents, or resolved using conventional techniques. The representation of the configuration of any carbon-carbon double bond appearing herein is selected for convenience only, and unless explicitly stated, is not intended to designate a particular configuration. Thus a carbon-carbon double bond depicted arbitrarily as E can be Z, E, or a mixture of the two in any proportion. Likewise, all tautomeric forms are also intended to be included.

The formulations include those suitable for oral, parenteral (including subcutaneous, intradermal, intramuscular, intravenous and intraarticular), rectal and topical (including dermal, buccal, sublingual and intraocular) administration. The most suitable route depends upon the condition and disorder of the recipient. The formulations can conveniently be presented in unit dosage form and can be prepared by any of the methods known in the art of pharmacy. All methods include the step of bringing into association at least one compound of the present disclosure or a pharmaceutically acceptable salt or solvate thereof (“active ingredient”) with the carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

The pharmaceutical preparations of the disclosure are preferably in a unit dosage form, and can be suitably packaged, for example in a box, blister, vial, bottle, sachet, ampoule or in any other suitable single-dose or multi-dose holder or container (which may be properly labeled); optionally with one or more leaflets containing product information and/or instructions for use. Such unit dosages generally contain between 1 and 1000 mg, and usually between 5 and 500 mg, of the at least one compound of the disclosure, e.g., about 10, 25, 50, 100, 200, 300 or 400 mg per unit dosage.

Formulations of the present disclosure suitable for oral administration can be presented as discrete units such as capsules, cachets or tablets each containing a predetermined amount of the active ingredient; as a powder (including micronized and nanoparticulate powders) or granules; as a solution or a suspension in an aqueous liquid or a non-aqueous liquid; or as an oil-in-water liquid emulsion or a water-in-oil liquid emulsion. The active ingredient can also be presented as a bolus, electuary or paste.

A tablet can be made by compression or molding, optionally with one or more accessory ingredients. Compressed tablets can be prepared by compressing in a suitable machine the active ingredient in a free-flowing form such as a powder or granules, optionally mixed with a binder, lubricant, inert diluent, lubricating, surface active or dispersing agent. Molded tablets can be made by molding in a suitable machine a mixture of the powdered compound moistened with an inert liquid diluent. The tablets can optionally be coated or scored and can be formulated so as to provide sustained, delayed or controlled release of the active ingredient therein.

The treatments (therapies) described herein can also be part of “combination therapies.” Combination therapy can be achieved by administering two or more agents, each of which is formulated and administered separately, or by administering two or more agents in a single formulation. The second active ingredient can be, for example, a second compound identified herein or through screens described herein, or active ingredients useful for treating, for example, depression, depressive-like symptoms, or symptoms associated with treatment by the first active agent (“side effects”). Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within any number of hours of each other or within any number or days or weeks of each other.

The present disclosure is also directed to kits for treating or preventing depression or depressive-like symptoms comprising compound(s) identified herein or compound(s) identified through the screening methods provided herein. The kits of the present disclosure can include, for example, components necessary for delivering a therapeutically effective amount of the active agent, instructions for use and/or devices for delivery of the active agent(s).

Astrocytes regulate responses to sleep deprivation, including changes in NREM slow wave activity (SWA), as well as increased sleep time and the cognitive impairments that follow sleep deprivation (Halassa, M. et al., Neuron, 61:213-219, 2009). Conditional expression of the SNARE domain of the vesicle protein VAMP2 selectively within astrocytes (dnSNARE mice (Pascual, O. et al., Science, 310:113-116, 2005)), causes reduced extracellular adenosine level as assessed by changes in the tonic activation of neuronal adenosine (A1) receptors as well as by biosensor measurements of adenosine. In the dnSNARE mouse, reduced activation of A1 receptors leads to a reduction in the pressure to sleep, as well as reduced electrophysiological responses to sleep deprivation.

Activation of the A1 receptor is involved in the homeostatic regulation of sleep (Basheer, R. et al., Prog. Neurobiol., 73:379-396, 2004). Adenosine accumulates as a function of prior wakefulness (Porkka-Heiskanen, T. et al., Neuroscience, 99:507-517, 2009). Introducing adenosine into the brain induces sleep (Strecker, R. et al., Behav. Brain Res., 115:183-204, 2000; Thakkar, M. et al., Neuroscience, 122:1107-1113, 2003) and the appearance of electrophysiological markers of homeostatic sleep pressure (Benington, J. et al., Brain Res., 692:79-85, 1995), whereas antagonizing adenosine by pharmacological agents promotes wakefulness (Snyder, S. et al., Proc. Natl. Acad. Sci. USA, 78:3260-3264, 1981) and attenuates the accumulation of homeostatic sleep pressure (Landolt, H., Biochem. Pharmacol., 75:2070-2079. 2008). Adenosine signaling is also implicated in the control of human sleep because of the powerful wake promoting effects of adenosine receptor antagonists (Landolt, H. et al., Neuropsychopharmacology, 29:1933-1939, 2004) and because humans with polymorphisms in the adenosine metabolizing enzyme, adenosine deaminase, show reduced adenosine metabolism and exhibit deeper sleep (Bachmann, V. et al., Cereb. Cortex, epub, 2011; Mazzotti, D. et al., Sleep, 34:399-402, 2011).

Disclosed herein are murine behavioral despair models of depression that effectively model the effects of sleep deprivation that occurs in the human population. In mouse models, 12 h of sleep deprivation reduces immobility in these behavioral tests. Also disclosed herein are the antidepressive-like effects of sleep deprivation, which require astrocytic signaling. Conditional expression of astrocytic dnSNARE, which reduces the activation of neuronal A1R, prevents antidepressive-like effects of sleep deprivation. The importance of A1R signaling in mediating the glial-dependent effects of sleep deprivation is further demonstrated by the observations that A1R^{-/-} mice and intracerebroventricular administration of an A1R antagonist prevent the positive effects of sleep deprivation. To determine whether A1R activation in the absence of sleep deprivation would lead to antidepressive effects, it was determined that i.c.v. administration of the A1R agonist, CCPA, promoted sleep and subsequently, after mice woke, antidepressive-like responses were observed. These responses slowly reversed over the subsequent five days. Together, these results demonstrate that glial-dependent A1R activation exerts powerful and immediate positive effects on depressive-like behaviors.

The present disclosure is also directed to models and screening methods useful for identifying A1R agonists that can be used to treat depression and depressive-like symptoms in addition to other conditions that suffer from pathological sleep perturbations, e.g., sleep disorders in the elderly, Parkinson's disease, Alzheimer's disease, epilepsy, schizophrenia, and symptoms experienced by recovering alcoholics. The compounds identified as A1R agonists can be validated for their efficacy in treating depression and depressive-like symptoms can be, for example, validated by using the mouse model and tests described herein, e.g., forced swim test and tail suspension test.

Astrocytes express receptors that raise intracellular Ca²⁺ levels, which, it has been discovered, leads to cellular adenosine release. Identifying compounds (ligands) that raise Ca²⁺ levels in astrocytes or astrocyte-based call lines, e.g., human astrocytoma cell lines, therefore, allows one of skill in the art to screen for ligands that stimulate cellular release of adenosine. One of skill in the art will recognize that compound identified as promoting increased Ca²⁺ levels can then be analyzed for their efficacy in increasing extracellular adenosine levels. One of skill in the art could validate the efficacy of such identified ligands by directly detecting extracellular adenosine levels in, for example, animal models or cultured cells. Alternatively, one of skill in the art could identify such ligands by directly evaluating test compounds for their ability to stimulate extracellular adenosine release in, for example, animal models or cultured cells.

If one or more compounds are identified as raising intracellular Ca²⁺ levels and extracellular adenosine levels, such identified compounds can then be evaluated for their efficacy in treating, for example, diseases, disorders or conditions characterized by pathological sleep perturbations including, but not limited to, depression or depressive-like symptoms, sleep disorders in the elderly, Parkinson's disease, Alzheimer's disease, epilepsy, schizophrenia and symptoms experienced by recovering alcoholics depression or depressive-like symptoms using the mouse models and, for example, the forced swim test and/or tail suspension test.

Intracellular Ca²⁺ levels can be measured, for example, by use of a marker that can detect Ca²⁺ in cells. Such a marker can be, for example, a fluorescent marker, a radiolabeled marker, a small molecule marker or an antigenic marker. In the case of fluorescent marker(s), cells with increased Ca²⁺ levels can be identified by fluoroscopic means including, but not limited to, fluorescent imaging, colorimetric assays and FACS analysis. In one embodiment, target compounds (ligands) can be tested for activation of astrocytic receptor-induced Ca²⁺ signals by measuring receptor-induced Ca²⁺ changes with the fluorescent Ca²⁺ indicator, fluo-4. Astrocytes are plated into 24-well plates, and, after one week, fluorometric measurements are made and responses of cells to a serial dilution of ligands are determined. Ca²⁺ fluorescence change can be determined, for example, by normalized accumulation of fluorescent change of three timepoints after ligand administration subtracted by value from an artificial cerebrospinal fluid (ACSF) control.

Marker and molecular sensors can also be used to measure extracellular adenosine levels. Electrochemical biosensors, for example, can be used to measure extracellular adenosine levels in situ using brain slices of mice exposed to a candidate compound.

Biosensor electrodes (Sarissa Biomedical) can be coated, for example, with an enzymatic matrix surrounding a platinum electrode (50 μm diameter), which is polarized to +500 mV. Electrochemical detection occurs via detection of hydrogen peroxide produced by the degradation reaction (Frenguelli, B. et al., J. Neurochem., 86:1506-1515, 2003). To control for electrical noise and non-specific electrochemical signal, two sets of biosensor can be employed. Adenosine biosensors (ADO) can be coated with an enzymatic layer containing, for example, nucleoside phosphorylase, xanthine oxidase and adenosine deaminase. As inosine (INO) biosensors lack adenosine deaminase, they are therefore insensitive to adenosine. Before use, all electrodes are hydrated and precalibrated with 10 μM adenosine in aCSF (124 mM NaCl, 26 mM NaHCO₃, 1 mM NaHPO₄, 10 mM Glucose, 1 mM sodium pyruvate, 2.9 mM KCl, 2 mM CaCl₂, 1 mM MgCl₂). Using these electrode biosensors for example, adenosine levels can be measured in situ in horizontal hippocampal slices. Post calibration using 10 μM adenosine standard can be used to scale the adenosine signal. Inosine at 10 μM can also be applied at the end of a screen to calibrate the relative sensitivity of INO and ADO biosensors to inosine. Estimation of tonic extracellular adenosine, for this method, for example, is determined using INO subtracted ADO signals following the 20 minute stabilization period. The value is scaled to the post-calibration standard to estimate concentration. Potentiostat-based recordings can be made using commercially available equipment, such as, for example, the ME200+ Duo-Stat (Sycopel International Ltd. Jarrow, UK), and digitized via, for example, a Digidata 1320 digitizer (Molecular Devices). Storage and analysis can be performed by methods known in the art, for example, by using Clampex 9.2 software (Molecular Devices). In one embodiment, during recording, slices can be perfused with aCSF at a rate of 1.2 mL/min with temperature maintained at 32.5-33 C.

To analyze biosensor data, one assumes that the sensitivity of both INO and ADO electrodes are linearly proportion to inosine and that the inosine biosensor is insensitive to adenosine. Raw signals are corrected to account for different sensitivities of the paired electrodes. The inosine signal, for example, can be corrected using the ratio of the sensitivity of the ADO and INO electrodes to the inosine standard. The resulting INO response is then subtracted from the ADO signal to obtain the current response specific to adenosine. Finally, this signal is calibrated to the adenosine standard to provide the concentration estimate. The capacitative discharge component in the raw signals can then be determined based on a fit of the signal measured in the slices chamber prior to placement of the electrodes and subtracted before scaling. Signal analysis can be performed using a variety of methods and tools known in the art, for example, SigmaPlot (Systat) and MATLAB® computing software (MathWorks, Natick Mass.).

Test compounds (ligands) identified by measuring Ca²⁺ and adenosine levels can be further tested, for example, by injecting them intracerebroventricularly into mice as described herein. Mice can be monitored using EEG and EMG to record vigilance states, and the power of slow wave activity, spindles, theta and gamma activity. The identified compounds can be tested, for example, to determine whether they increase total sleep time, as well as reduce sleep fragmentation and increase the power of slow wave activity during NREM sleep.

One of skill in the art will appreciate that cannula can be implanted in mice. For example, for intracerebroventricular cannula (Plastics One) implantation, mice can be deeply anaesthetized and placed in a stereotaxic apparatus. Following opening, a hole is drilled at coordinates −1.0 mm AP, −1.0 mm ML relative to bregma for insertion of the cannula, and two additional holes are drilled nearby for anchoring screws. Cannulae are lowered into position using a stereotaxic attachment, and secured in place using dental cement. Following curing of the cement, the animal is sutured and allowed to recover for a minimum of one week before infusion and/or behavioral testing.

One of skill in the art would know how to monitor mice using, for example, EEG and EMG. For electroencephalography, mice can be, for example, implanted with EEG and EMG electrodes under ketamine/xylazine anesthesia. After 5-7 days of post-operative recovery, lightweight recording cables can be connected to the head implants and mice placed in cylindrical polypropylene containers containing nest material, water and food ad libitum. Mice are allowed to acclimated to the sleep chamber for 5-7 days (12:12 light-dark (LD) cycle; lights on at 8 AM). EEGs and EMGs can be collected using commercially available instruments, such as, for example, a Pinnacle Technologies system (digitized at 1000 Hz). Following acclimation, baseline 24-h recordings can be made.

Vigilance state scoring and analysis can be performed by monitoring sleep states of mice. For example, NonREM sleep, REM sleep and wake are determined by an experimenter blind to experimental condition. On the baseline day, the amount of each state is computed (expressed as a percentage of total recording time) and the duration of individual sleep and wake episodes. Transitions between NREM sleep and REM sleep during the light phase are measured. To investigate the beneficial effects of sleep deprivation in mice, sleep deprived (SD) mice, from 8 AM to 8 PM, are monitored at the beginning of the major sleep period (light phase) using, for example, the Pinnacle sleep deprivation chamber.

Described herein are methods to determine whether candidate ligands, which were injected intracerebroventirucularly, promote antidepressive-like actions when mice are subsequently tested in the forced swim and tail suspension tests of behavioral despair, as well as using the sucrose consumption test for anhedonia. These tests are performed in wild type and dnSNARE mice, which contain a genetic inhibition of the astrocytic adenosine pathway. Positive effects of candidate compounds are prevented in dnSNARE but not wildtype mice.

The forced swim test is typically performed in a quiet experimental room as described in previous reports (Porsolt, R. et al., Arch. Int. Pharmacodyn. Ther., 229:327-336, 1977). For a typical test, animals are placed, for example, into a plexiglass cylinder (25 cm height, 25 cm diameter) containing 20 cm height of water at 23-25 C for 10 min. The animals typically display very fast swimming initially, lasting 1-2 min, followed by increasing immobility. Immobile time is measured during the last 4 min of the total period for statistical analysis. The animals are videotaped and movements are digitized from above. Immobility can be automatically scored, for example, using Ethovision (Noldus Information Technology Inc., Leesburg, U.S.A.). Results from tracking analysis can be analyzed using, for example, ANOVA to compare means. Predictive validity of the forced swim test can be confirmed with imipramine in a separate experiment.

The tail suspension test can be performed in a quiet experimental room as described in previous reports (Bai, F. et al., Pharmacol. Biochem. Behav., 70:187-192, 2001). For a typical test, each mouse is suspended by its tail to a horizontal wooden bar located inside a white plastic box (40 cm×46 cm×40 cm) approximately 35 cm above the floor. The mouse is secured to the bar by adhesive tape placed 1-1.5 cm from the tip of the tail, such that the mouse's head is about 20 cm above the floor. The trial is conducted for 6 min (360 s) during which time the behavior is video recorded followed by digitizing and automated scoring using, for example, Ethovision. Results from tracking analysis can be analyzed, for example, using ANOVA to compare means. When mice are observed to climb their tails (>10% of total time) they are eliminated from further analyses.

Sucrose consumption experiments can be conducted known to one of skill in the art. For a typical test, mice are gradually acclimatized to increasing durations of water deprivation (0, 4, 8, 12, and 16 hours) and i.c.v. infusion of saline over the course of five days to minimize the stressfulness of the testing. On the sixth day, instead of receiving water at the end of the deprivation period, mice are given a 1% v/w solution of sucrose. This is repeated for three days (days 6-9) to establish baseline sucrose consumption levels. During the deprivation period preceding day 10, mice are given i.c.v. infusions of the identified test compound, followed by the presentation of sucrose as during baseline consumption testing. The average baseline (saline infusion) sucrose consumption is then compared to the average sucrose consumption following test compound infusion. Results are analyzed using a Student's t-test to compare means.

Mice can also be monitored, for example, by an open field test, as performed in accordance with the EMPReSS resource phenotyping protocol (see Example).

The findings described herein demonstrate that the activation of astrocyte-specific receptors leads to a modulation of extracellular adenosine. One of skill in the art would recognize that receptor agonists can be developed as therapeutic agents that act on these receptors to control adenosine and potential depressive like behaviors without the peripheral side effects of adenosine pharmacology.

### EXEMPLIFICATION

### Example 1

Described herein are results of investigating the mechanisms underlying the robust improvement observed in human depression patients following sleep deprivation. Using pharmacology and molecular genetics, astrocyte-to-neuron signaling pathways were modulated, and the beneficial effects of sleep deprivation on depressive-like behaviors are shown to require an astrocyte-dependent signaling pathway. Using animal models with the knowledge that sleep deprivation is highly effective for treating human depression, the critical importance of A1R signaling for these beneficial effects is herein demonstrated. A1R receptors are required since A1R^{-/-} mice as well as mice in which the A1R antagonist CPT was delivered centrally, both fail to respond to sleep deprivation with reduced immobility in the forced swim and tail suspension tests. Additional support for A1R signaling mediating antidepressive effects are provided by the observation that sustained sleep deprivation (72 h) leads to both an inactivation of the A1R pathway and to a loss of the antidepressive effects of sleep deprivation. Since exogenous, central activation of A1R using CCPA led to antidepressive effects, glial-derived adenosine acting through A1R mediates the antidepressive effects of sleep deprivation.

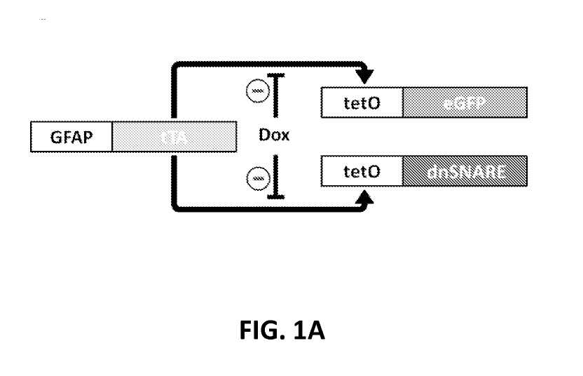

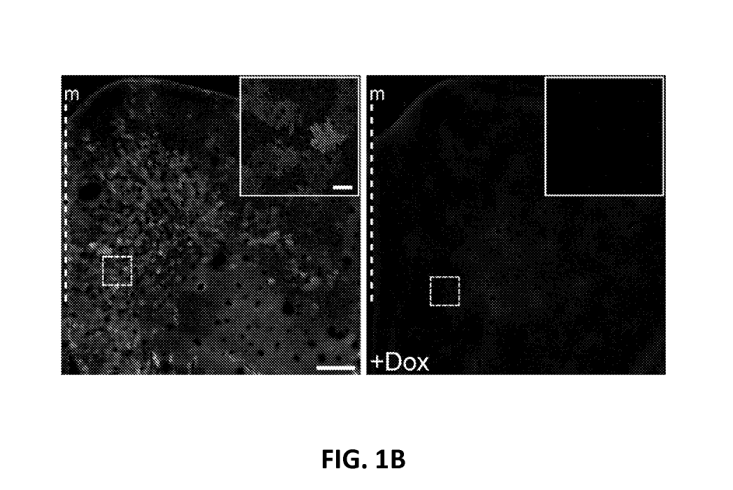

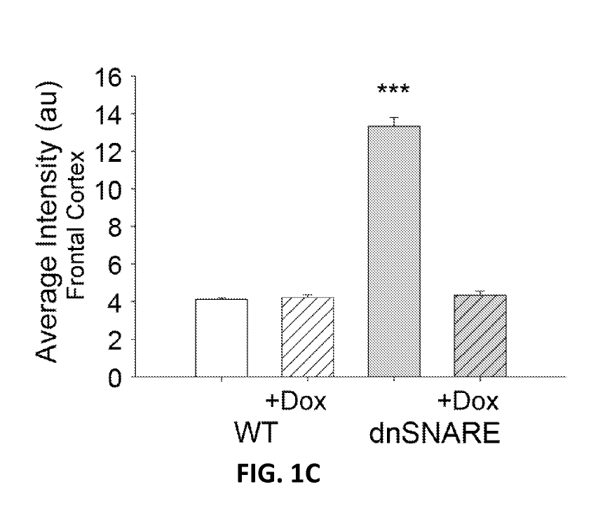

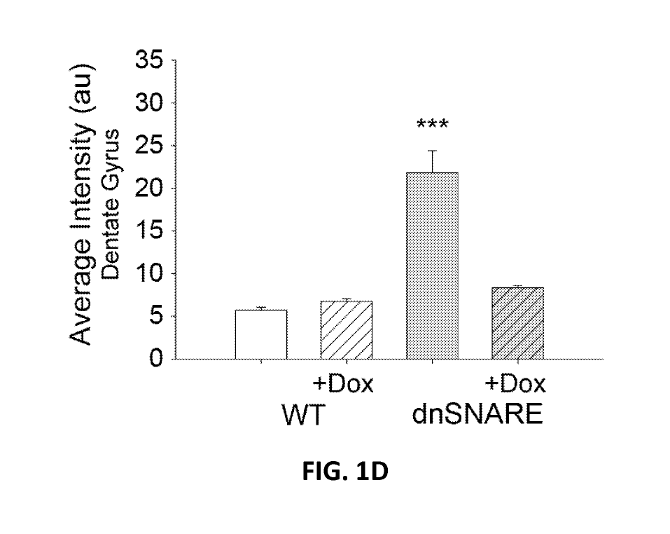

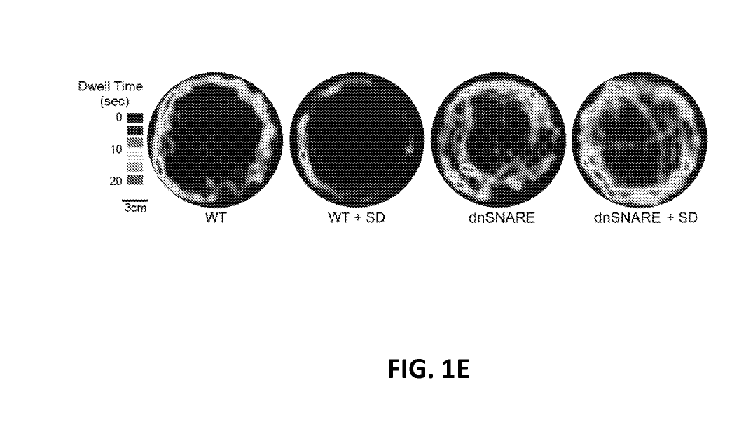

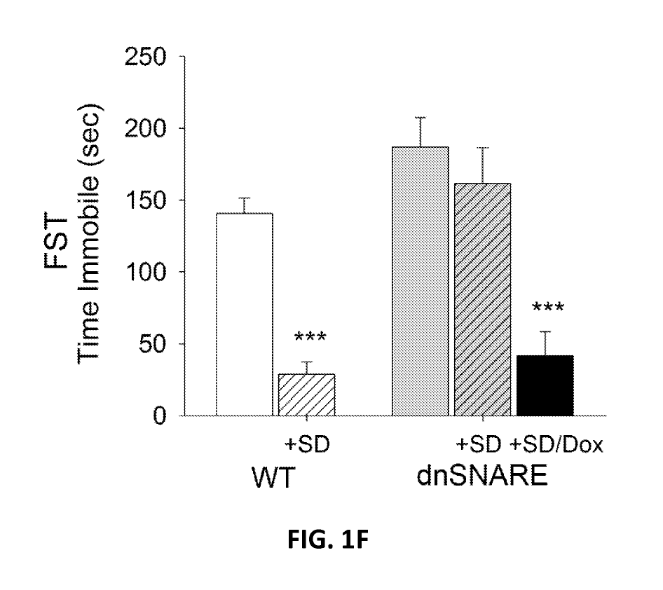

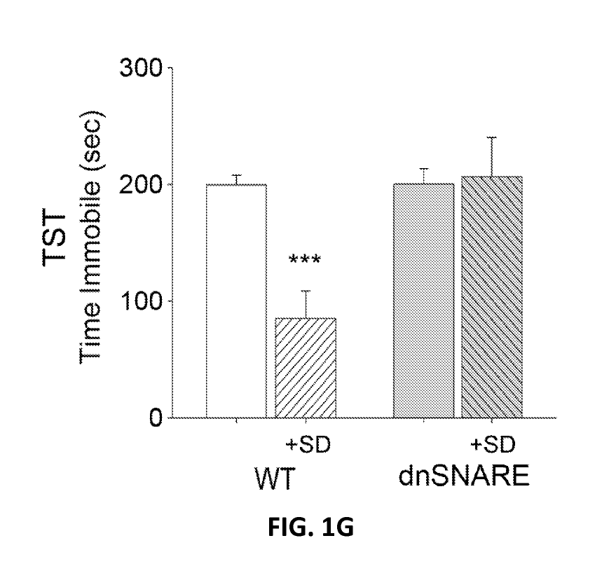

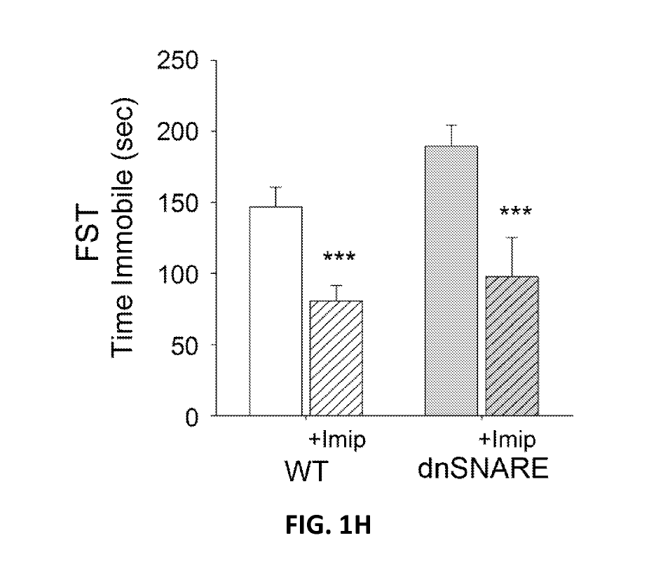

Animal models of specific symptoms of human depression have proven to be essential tools in revealing the mechanisms of psychiatric diseases. In relation to depression, two tests that are well characterized, robust and respond to many antidepressive drugs are the forced swim test and the tail suspension test. The strain of mice used in this study, C57Bl6, has been consistently shown to have high levels of depressive-like behavior compared to other inbred strains. Before interpreting changes in immobility in these two behavioral despair tasks as being relevant to depression, further validation is provided by showing that 14 days of treatment with a tricyclic antidepressant, imipramine, reduces immobility (FIG. 1H) (Fukui, M. et al., J. Neurosci., 27:10520-9, 2007; Sanchez, C. et al., Psychopharmacology (Berl.), 129:197-205, 1997), and that sleep deprivation, a powerful and rapid acting antidepressant treatment in the clinical population, reduces immobility in these tasks. Final validation was provided by an additional behavioral test of anhedonia that was performed using the sucrose consumption test. It is important when studying animal models to use multiple tasks to insure validity. Taken together this validation shows that activation of the glial-dependent adenosine pathway contributes to antidepressive effects of sleep deprivation.

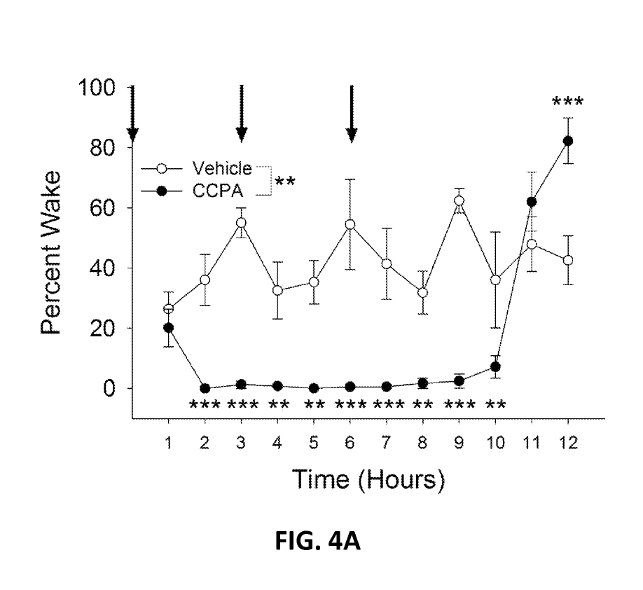

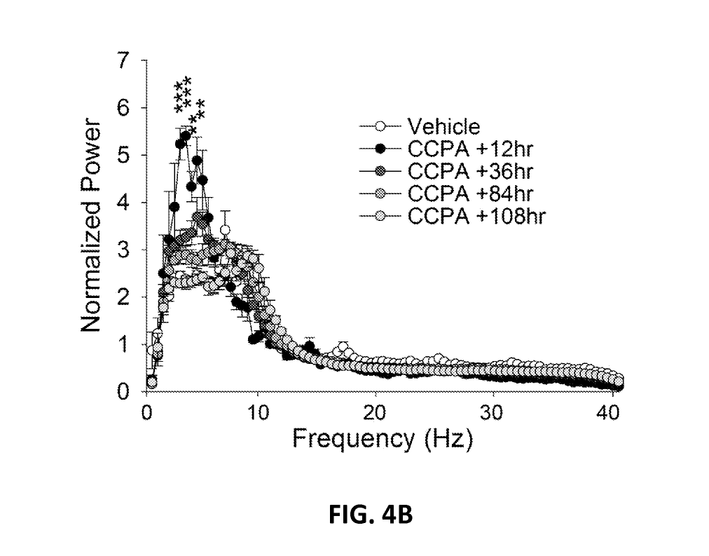

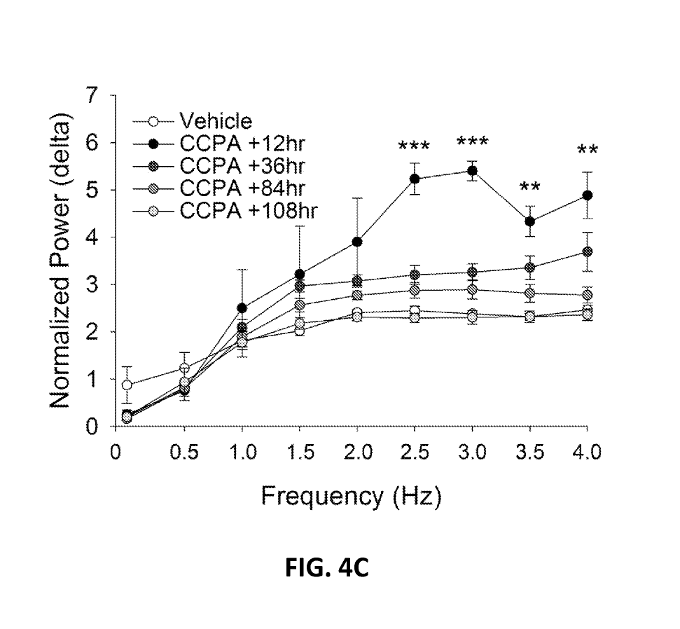

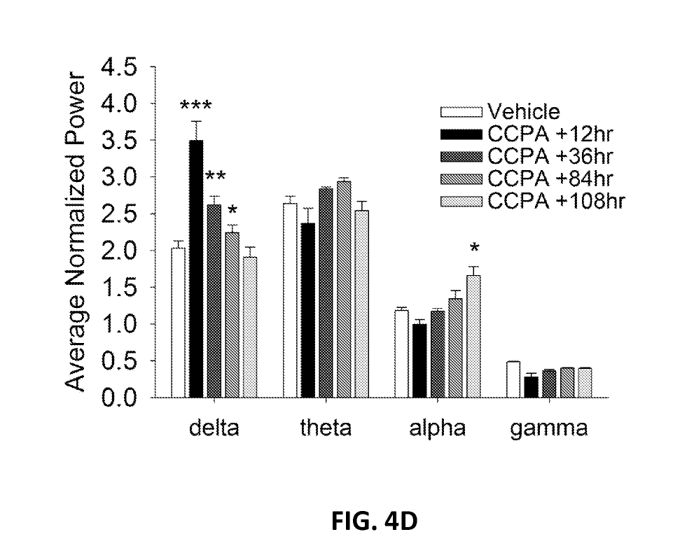

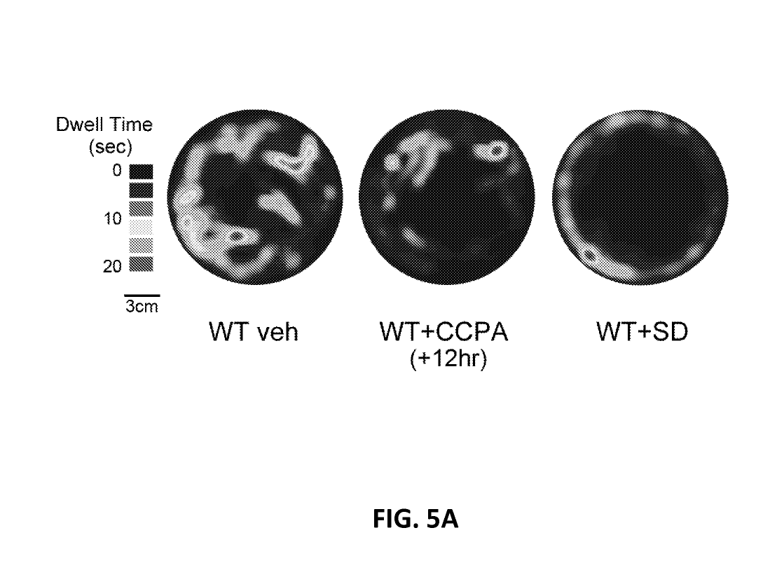

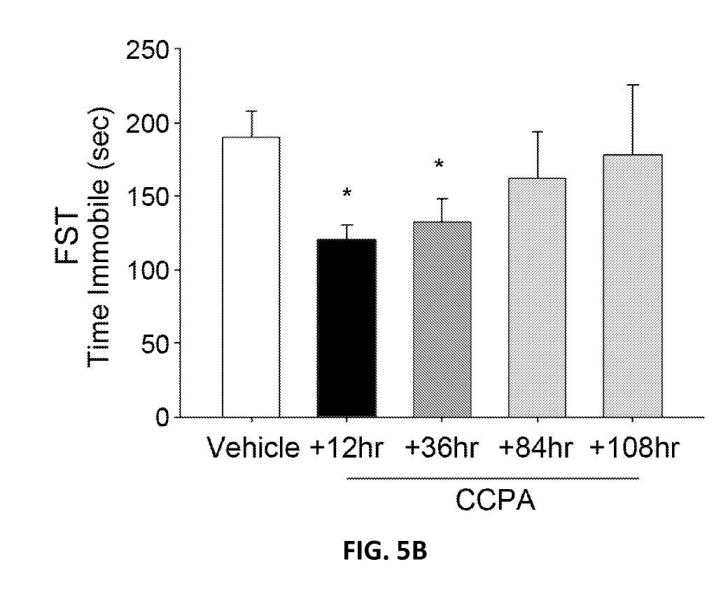

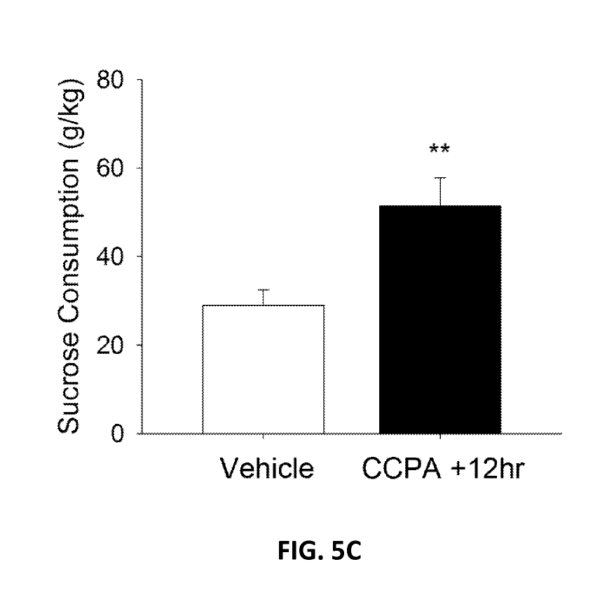

Whether antidepressive effects of A1R activation converge on pathways recruited by other treatments is unknown. However, there are common observations concerning the modulation of delta power of the EEG. EEG measurements revealed a significant increase in delta power during wakefulness that decayed in a temporally coincident manner with the antidepressive effects of this ligand: CCPA caused a significant increase in delta power for up to 36 hours following onset of administration of this A1R ligand (FIGS. 4B-D) and antidepressive effects for the same time period (FIG. 5B). Other fast acting antidepressant treatments are also known to increase delta power including ketamine (Murrough, J. et al., J. Clin. Psychiatry, 72:414-5, 2011; Freye, E. et al., Neurophysiol. Clin., 35:25-32, 2005), deep brain stimulation (Kennedy, S. et al., Am. J. Psychiatry, 168:502-10, 2011; Mayberg, H. et al., Neuron, 45:651-60, 2005), transcranial magnetic stimulation (Spronk, D. et al., Clin. EEG Neurosci., 39:118-24, 2008; Griskova, I. et al., Neurosci. Lett., 419:162-7, 2007) and electroconvulsive therapy (Fink, M. & Kahn, R., AMA Arch. Neurol. Psychiatry, 78:516-25, 1957; Sackeim, H. et al., Arch. Gen. Psychiatry, 53:814-24, 1966) raising the possibility of convergent mechanisms of action.

### Animals and Genetic Strains

All procedures conducted were in strict accordance with the National Institutes of Health Guide for the Care and Use of Laboratory Animals, and were approved by Tufts University and the Institutional Animal Care and Use Committee. The dnSNARE and A1R^{-/-} mouse strains were previously characterized. Germ-line transmission of the transgenes was detected using PCR to identify all experimental animals. Both the dnSNARE and A1R^{-/-} mice had been backcrossed onto a C57BL6/J genotype for more than 10 generations. Consequently, dnSNARE and A1R^{-/-} littermates were used as controls.

### Sleep Deprivation

Sleep deprivation of the mice is achieved using the Automated Sleep Deprivation System for Mice (Pinnacle technologies). The apparatus is composed of an adjustable metal bar that is positioned above the bottom of the cage and rotates during periods of sleep. The mouse must wake up to move around the bar as it rotates, and rotation of the bar ceases once the animal has been awake for 30 seconds, limiting exercise and stress effects. Preliminary studies have shown this to be an effective method for long-term sleep fragmentation and/or deprivation.

### Forced Swim Test (FST)

The forced swim test (FST) was performed in a quiet experimental room. Animals where placed into a Plexiglass cylinder (25 cm height, 25 cm diameter) containing 20 cm height of water at 23-25° C. for 10 min. The animals typically displayed very fast swimming initially, lasting 1-2 min, followed by increasing immobility. Immobile time was measured during the last 4 min of the total period for statistical analysis. The animals were videotaped and movements were digitized from above. Immobility was automatically scored using Ethovision (Noldus Information Technology Inc., Leesburg, U.S.A.). Results from tracking analysis were analyzed using ANOVA to compare means. Predictive validity of the FST was confirmed with imipramine in a separate experiment.

### Tail Suspension Test (TST)

The tail suspension test was performed in a quiet experimental room as described in previous reports. Each mouse was suspended by its tail to a horizontal wooden bar located inside a white plastic box (40 cm×46 cm×40 cm) approximately 35 cm above the floor. The mouse was secured to the bar by adhesive tape placed 1-1.5 cm from the tip of the tail, such that the mouse's head was about 20 cm above the floor. The trial was conducted for 6 min (360 s) during which the behavior was video recorded followed by digitizing and automated scoring using Ethovision. Results from tracking analysis were analyzed using ANOVA to compare means. When mice where observed to climb their tails (>10% of total time) they were eliminated from further analyses.

### Sucrose Consumption

Sucrose consumption was conducted (Papp, M. et al., Psychopharmacology (Berl.), 104:255-9, 1991). After the acclimatization and habituation period mice were given access to sucrose for three days (days 6-9) to establish baseline sucrose consumption levels. Mice were then given i.c.v. infusions of CCPA administered as a series of three bolus injections at ZT=0, 3 and 6, followed by the presentation of sucrose (day 10). The baseline (vehicle infusion) sucrose consumption (day 9) was then compared to the sucrose consumption following CCPA infusion (day 10).

### Open Field Behavior

The open field test was performed in accordance with the EMPReSS resource phenotyping protocol (Brown, S. et al., Nat. Genet., 37:1155, 2005). Open field behavior of mice was videorecorded from above, and assessed using Ethovision. Results from tracking analysis were analyzed using ANOVA to compare means.

### Rotorod

The rotorod used a rotating cylinder that was 4 cm in diameter, fixed 35 cm above the ground and enclosed by transparent Plexiglas. The rotorod cylinder was covered in textured rubber coating, which facilitates traction. A small electric motor provided power to turn the rotorod via a rubber belt. The rotorod was set to rotate once every 5 s.

### Intracerebroventricular Cannula Implantation

For cannula (Plastics One) implantation, mice were deeply anaesthetized and placed in a stereotaxic apparatus. Following opening, a hole was drilled at coordinates −1.0 mm AP, −1.0 mm ML relative to bregma for insertion of the cannula, and two additional holes were drilled nearby for anchoring screws. Cannulae were lowered into position using a stereotaxic attachment and secured in place using dental cement. Following curing of the cement, the animal was sutured and allowed to recover for a minimum of one week before infusion and/or behavioral testing.

### Histology and Microscopy

For histology and immunohistochemistry, animals were transcardially perfused with a 4% solution of paraformaldehyde in PBS followed by cryoprotection in 30% sucrose. Brains were then flash frozen and cut using a sliding microtome to a thickness of 40 μm. To evaluate EGFP signal in dnSNARE mice, sections were mounted on slides and cover slipped with fluoromount G prior to confocal microscopy on a Nikon Ti confocal microscope. To detect A1R, ABC-DAB immunohistochemistry was used. For some experiments, endogenous peroxidases were blocked using a 0.3% H₂O₂ solution, followed by treatment with a sodium metaperiodate/lysine solution. After washing in PBS, sections were incubated in a blocking solution containing 2% normal goat serum, 4% bovine serum albumin, and 0.03% triton-X in PBS. Sections were incubated with the A1R antibody (1:5000 in blocking solution) overnight at 4 C. Sections were washed in PBS and incubated with biotinylated goat anti-rabbit secondary antibody, followed by avidin-biotin conjugate (Vector ABC kit), and visualization with DAB (Vector DAB kit). Stained sections were mounted on slides, allowed to dry overnight, dehydrated in a series of alcohol and embedded in entallan out of xylene. Sections were visualized using a light microscope.

### Preparation and Extracellular Recording of CA1 Neurons in Acute Hippocampal Slice

Mice were briefly anaesthetized with isofluorane and then decapitated, and slices were prepared (Manzoni, O. et al., Science, 265:2098-101, 1994). The brain was rapidly extracted in cold cutting solution (124 mM NaCl, 26 mM NaHCO₃, 1 mM NaHPO₄, 10 mM Glucose, 1 mM sodium pyruvate, 0.6 mM ascorbate, 2.9 mM KCl, 1 mM CaCl₂, 2 mM MgCl₂) bubbled continuously with a 95% O₂ 5% CO₂ gas mixture. Slices 310 μm thick were cut using a Leica VT 1000S Vibratome. Isolated hippocampal slices were transferred to a bath containing continuously oxygenated cutting solution and incubated at 30 C for a 1.5 hour recovery period. Extracellular recordings were conducted in artificial cerebrospinal fluid (aCSF, 124 mM NaCl, 26 mM NaHCO₃, 1 mM NaHPO₄, 10 mM Glucose, 1 mM sodium pyruvate, 2.9 mM KCl, 2 mM CaCl₂, 1 mM MgCl₂) continuously superfused at 1.2 mL/minute and maintained at 32.8 C. Extracellular stimulation were delivered using a 125 μm concentric Pt—Ir electrode in unipolar mode with a pulse width of 0.1 ms.

### Electroencephalography

Mice were implanted with EEG and EMG electrodes under ketamine/xylazine anesthesia. After 5-7 days of post-operative recovery, lightweight recording cables were connected to the head implants and mice were placed in cylindrical polypropylene containers containing nest material, water and food ad libitum. Mice were acclimated to the sleep chamber for 5-7 days (12:12 light-dark (LD) cycle; lights on at 8 AM). EEGs and EMGs were then collected on a Pinnacle Technologies system and digitized at 1000 Hz. Following acclimation, baseline 24 h recordings were made.

### Vigilance State Scoring and Analyses

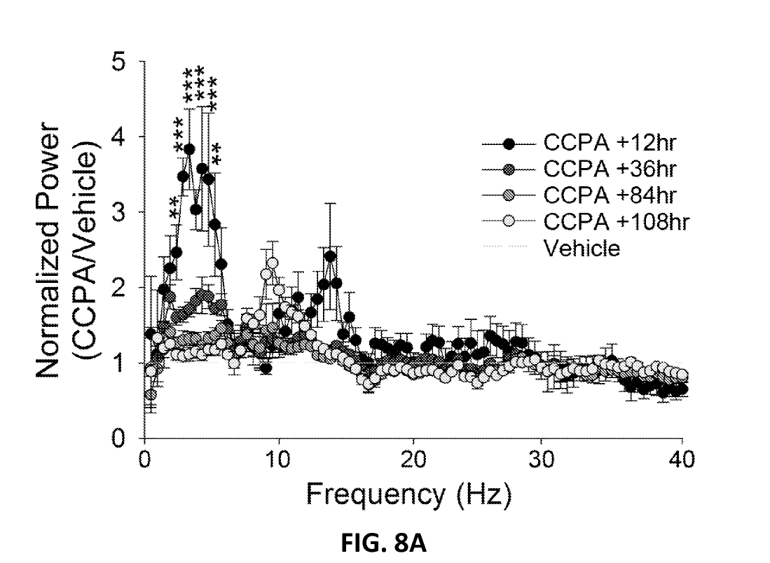

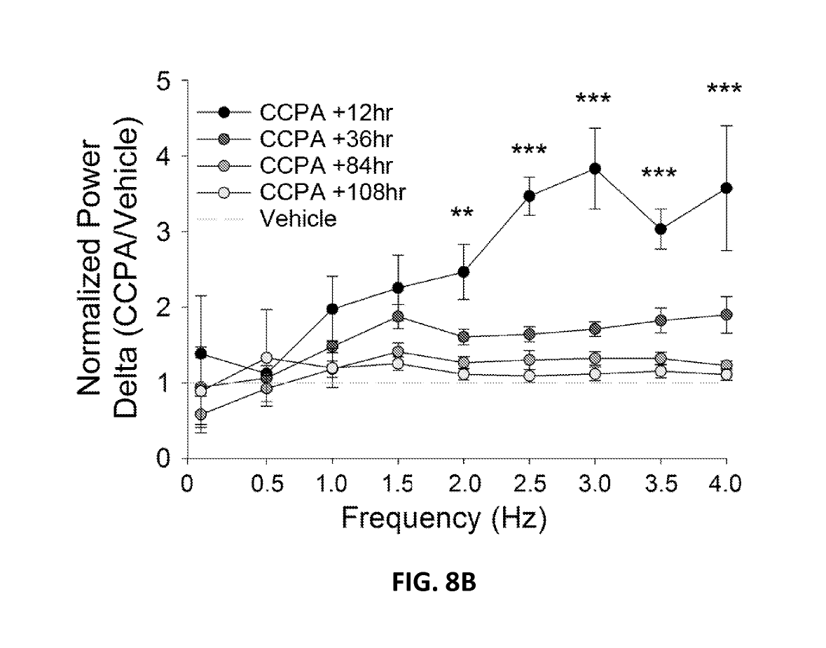

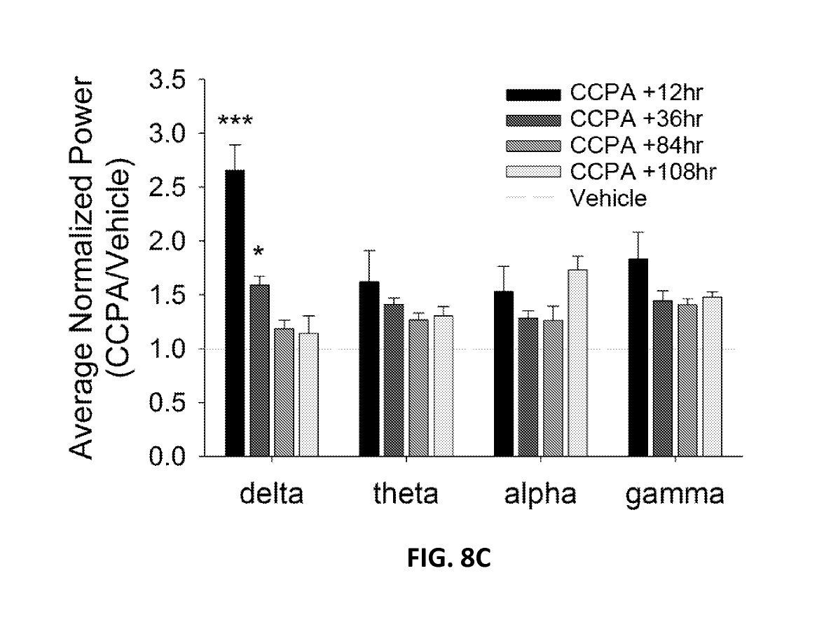

NonREM sleep (“NREM” sleep), REM sleep and wake were determined by an experimenter blind to experimental condition. On the baseline day, the amount of each state (expressed as a percentage of total recording time) and the duration of individual sleep and wake episodes were computed. Transitions between NREM sleep and REM sleep were also measured during the light phase. To investigate sleep the beneficial effects of sleep deprivation mice, mice were sleep deprived (SD) from 8 a.m. to 8 p.m. at the beginning of the major sleep period (light phase) using a Pinnacle sleep deprivation chamber. All EEG data were analyzed by normalizing each data point to the average power of the EEG from 0.5 to 40 Hz (FIG. 4), and by normalizing each data point (+12 hr, +36 hr, +84 hr, +108 hr) to the corresponding vehicle data points (FIG. 8).

### Statistical Analysis

Data were expressed as mean±s.e.m. and the statistical significance of differences in mean values was assessed by t-test (sucrose consumption), or analysis of variance (ANOVA; two way (Fluorescence quantification, FST, TST, EEG) or repeated measures (EEG FFT)) with Bonferroni post hoc comparison, as appropriate. Differences among means were considered significant at values of *: p≦0.05, **: p≦0.01, ***: p≦0.001.

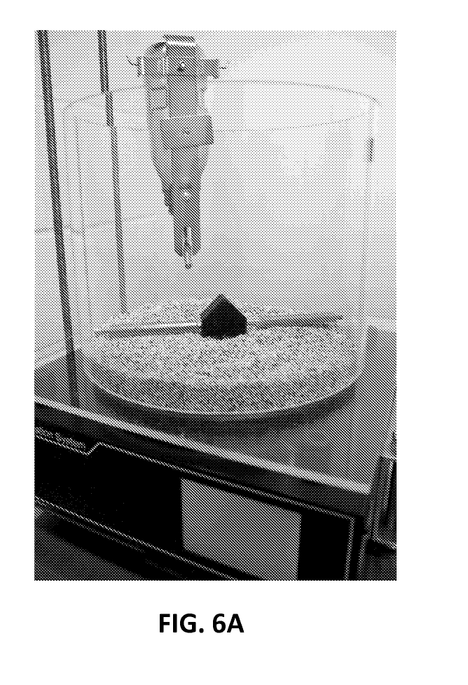

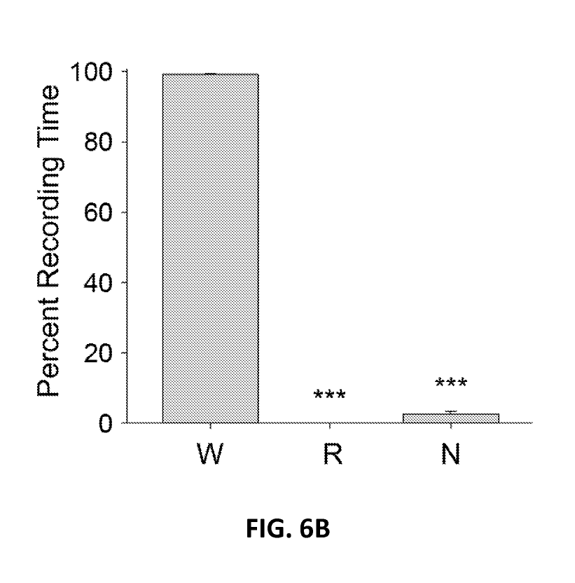

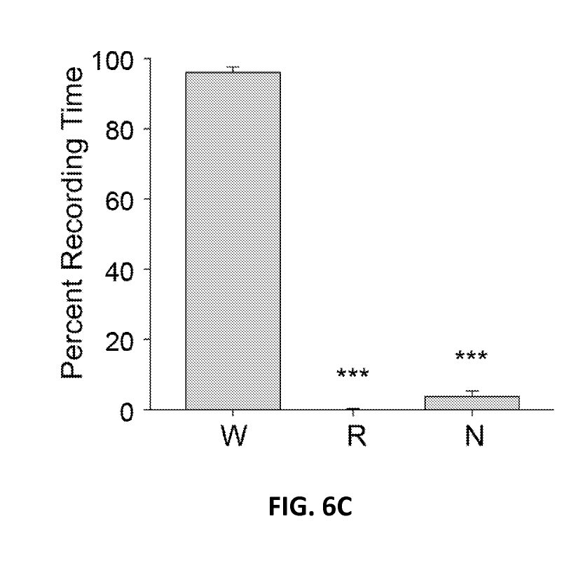

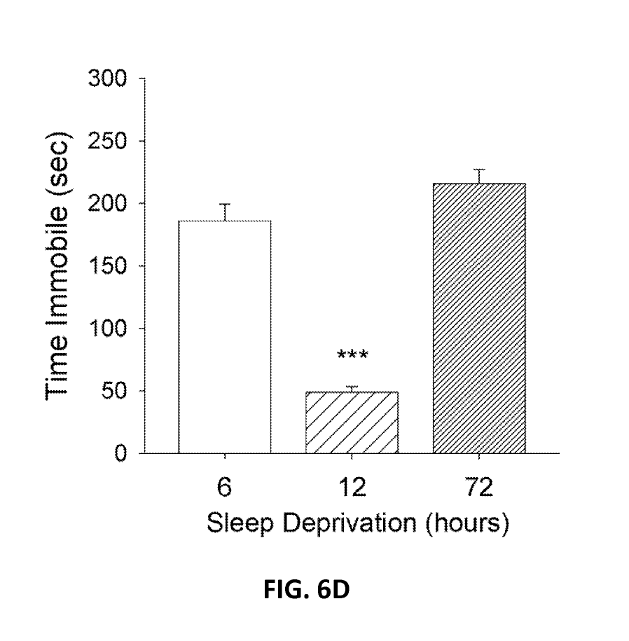

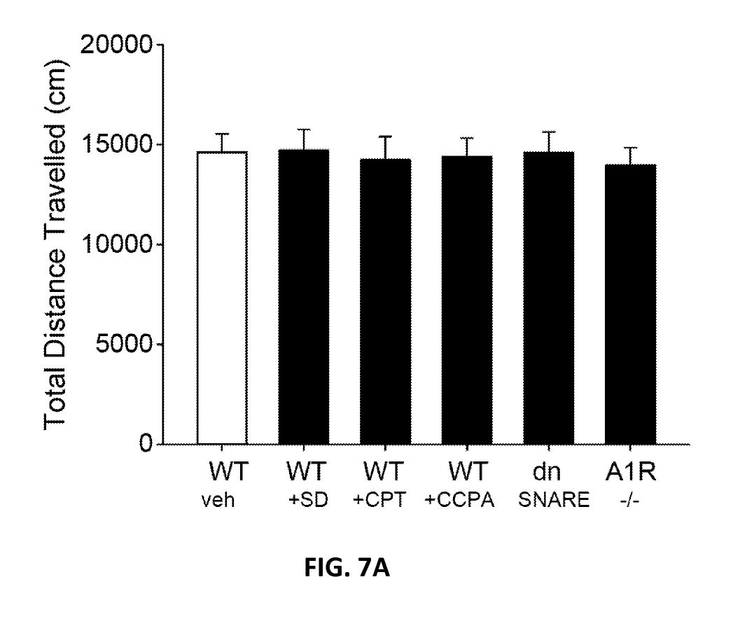

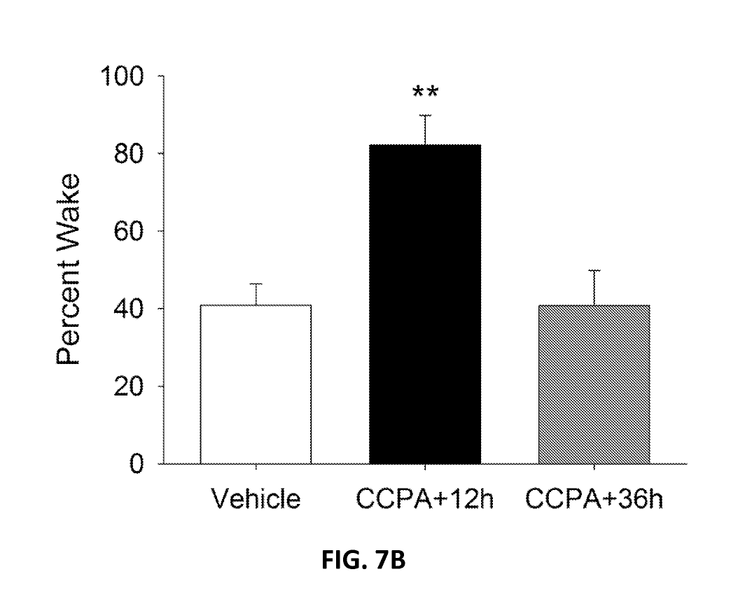

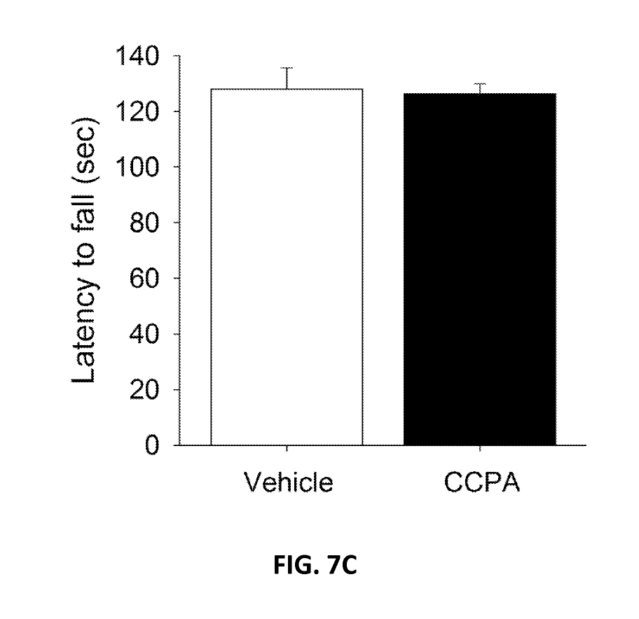

C57Bl/6J mice are considered to exhibit depressive-like behaviors as measured in multiple models of behavioral despair (Bai, F. et al., Pharmacol. Biochem. Behav., 70:187-192, 2001; Cryan, J. and Mombereau, C., Mol. Psychiatry, 9:326-357, 2004; Pothion, S. et al., Behav. Brain Res., 155:135-146, 2004; Miller, B. et al., PLoS One, 5:e14458, 2010). The use of C57Bl/6J mice and the behavioral despair tests to screen for therapeutic benefits resulting from sleep deprivation was validated by subjecting mice to different durations of sleep deprivation prior to a single period of either the forced swim test or the tail suspension test. Mice were subjected to sleep deprivation for 0, 12 or 72 h using a rotating bar in their home cage. EEG/EMG analyses confirmed that sleep deprivation was performed effectively (FIGS. 6A and 6B). In agreement with the human studies (Wu, J. & Bunney, W., Am. J. Psychiatry, 147:14-21, 1990), 12 h but not 72 h of sleep deprivation led to antidepressive-like effects revealed by a reduced immobility time in both the forced swim test and tail suspension test models of depression (FIG. 6C). Sleep deprivation reduced immobility time in the forced swim test from 214.97±8.47 s to 43.25±8.55 s (p<0.001) and in the tail suspension test from 199.73±3.75 s to 85.33±14.76 s (p<0.001) without accompanying changes in locomotor activity as measure in the open field (FIG. 7).

Astrocytes contribute to the behavioral and physiological responses to sleep deprivation. Astrocytes release chemical signals that modulate neurotransmission in a process termed gliotransmission (Haydon, P., Nat. Rev. Neurosci., 2:185-93, 2001). Gliotransmission is impaired in dnSNARE mice via conditional astrocyte-selective expression of the SNARE domain of the vesicle protein VAMP2. Conditional astrocyte-selective expression of the SNARE domain of the vesicle protein, VAMP2 (dnSNARE), attenuates SWA of NREM sleep and impairs compensatory increases in sleep time that follow sleep deprivation, actions that are mediated by the ability of the astrocyte to regulate extracellular adenosine. In the dnSNARE strain of mice the tTA/tetO system is used to drive expression of the reporter gene GFP and dnSNARE under control of the GFAP promoter (FIG. 1A). Animals were maintained on doxycycline (dox) to suppress transgene expression until weaning allowing for normal brain development (Fellin, T. et al., Proc. Natl. Acad. Sci. USA, 106:15037-15042, 2009). GFP reporter expression occurs selectively in astrocytes using antibodies to GFAP (astrocyte marker) and NeuN (neuronal cell marker). Confocal imaging and quantification of GFP signal revealed expression of transgenes in regions of the nervous system that are thought to be important in contributing to depressive symptoms including the frontal cortex (13.33±0.46 au; FIGS. 1B and 1C), and hippocampus (21.82±2.56 au; FIG. 1D).

Adenosine signaling is implicated in the control of human sleep, and humans with polymorphisms in the adenosine metabolizing enzyme, adenosine deaminase, show reduced adenosine metabolism and exhibit more consolidated sleep. Magnetic resonance spectroscopy has shown that brain purine levels are low in categories of depressed patients, suggesting that increasing brain adenosine levels may have antidepressive effects (Renshaw, P. et al., Am. J. Psychiatry, 158:2048-55, 2001). Additionally, compromised adenosine transport due to polymorphism(s) in the nucleoside transporter gene SLC29A3 have been identified in female patients predisposed to depression (Gass, N. et al., J. Affect. Disord., 126:134-9, 2010).

The effects of sleep deprivation were examined with forced swim test and tail suspension test in dnSNARE mice relative to wildtype (WT) littermate controls. The expression of astrocytic dnSNARE impaired the ability of 12 h of sleep deprivation to reduce immobility time in both the forced swim test and tail suspension test (FIGS. 1E-G). Mobility of animals in the forced swim test can be represented as density plots over time. Increased time spent in a particular position (immobility) is shown by red and orange (hot) colors, with less time spent (mobility) shown in blue (cold) colors. In the forced swim test, sleep deprivation reduced immobility time from 146.83±7.21 s to 38.47±9.13 s (p<0.001) in wildtype mice while in dnSNARE mice basal immobility (208.48±11.36 s), was not significantly affected by sleep deprivation (152.91±21.73 s; p=0.56).

Similarly using the tail suspension test, basal immobility in dnSNARE mice (200.33±8.30 s) was not significantly reduced by sleep deprivation (206.67±21.31 s; p=0.748). In contrast, when dnSNARE mice are maintained on a doxycycline diet to suppress expression of the dnSNARE transgene, sleep deprivation does significantly decrease immobile time using the forced swim test (dnSNARE: 315.74±5.52 s; dnSNARE+dox: 58.35±3.60 s). Because parallel experiments did not find an impact of sleep deprivation or of dnSNARE expression on locomotor activity (FIG. 7) these results indicate the astrocyte-dependent dnSNARE sensitive sleep homeostasis pathway contributes to antidepressive-like effects of sleep deprivation.

To further validate the results of the forced swim test and to ask whether the astrocytic SNARE-dependent pathway converges on imipramine (tricylcic antidepressant) sensitive systems, the influence of imipramine treatment on immobility time of dnSNARE mice and WT C57Bl/6J littermate controls was tested. Chronic administration of imipramine (14 days) was used to determine its effect on immobility time. Both wildtype littermates (saline: 149.42±4.62 s; imipramine: 73.00±9.53 s; p<0.001) and dnSNARE mice (saline: 196.27±8.84 s; imipramine: 88.47±20.10; p<0.001) showed a significant reduction in time spent immobile following chronic treatment (14 days) with imipramine (20 mg/kg; FIG. 1H). Because immobility time of wildtype and dnSNARE mice was not differentially affected by imipramine treatment (p=0.417), the astrocytic signaling plays a novel role in contributing to the beneficial effects of sleep deprivation on depressive-like symptoms.

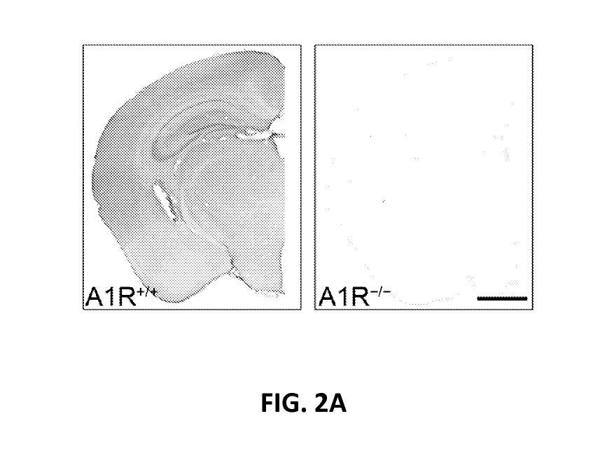

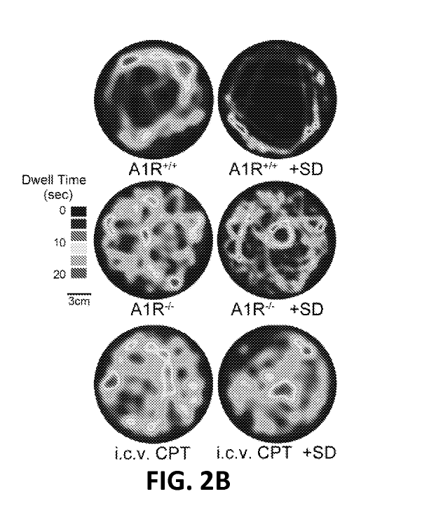

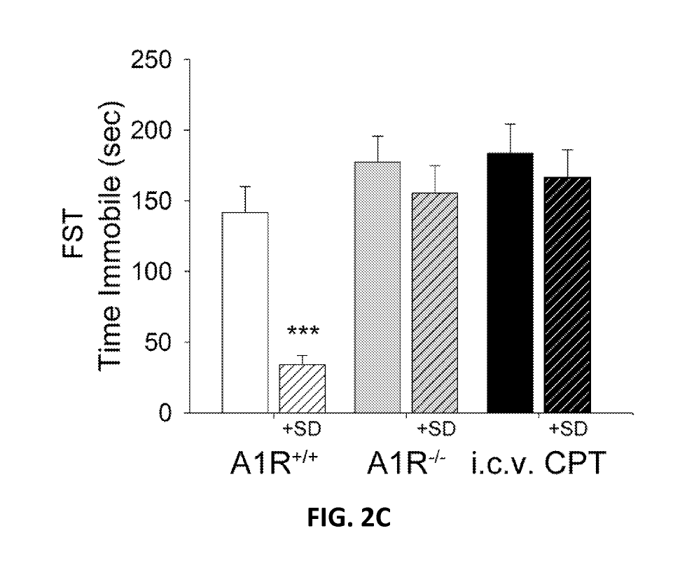

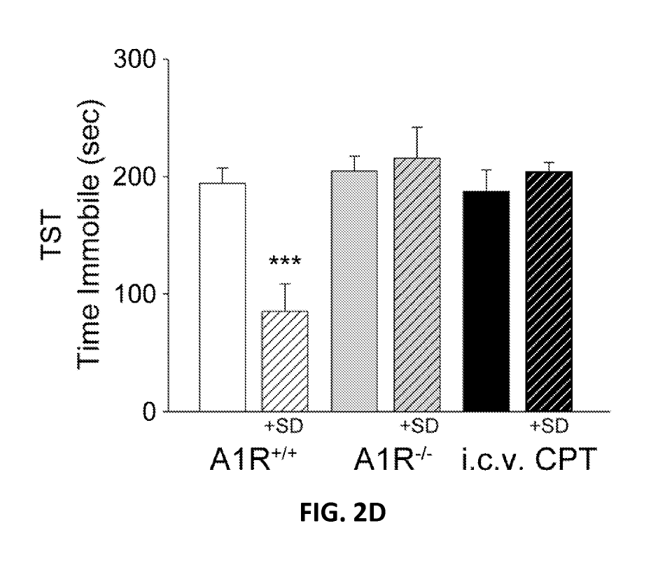

To determine whether the astrocytic SNARE-dependent signaling pathway is required for the beneficial effects of sleep deprivation on depressive-like behavior, genetic and pharmacological strategies were used. Mice in which the entire A1R coding sequence was deleted by homologous recombination (A1R^{-/-}; Sun, D. et al., Proc. Natl. Acad. Sci. USA, 98:9983-9988, 2001) were used. A1R^{-/-} mice do not express A1R mRNA transcripts and do not respond to A1R agonists, but are viable and do not exhibit gross anatomical abnormalities. A1R^{-/-} mice and wild-type littermates maintained with a C57Bl6/J genetic background (FIG. 2A) were subjected to 12 h of sleep deprivation. Independent groups of mice were subsequently evaluated in both the forced swim test and tail suspension test assays. Sleep deprivation failed to reduce the immobility time of A1R^{-/-} mice in either the forced swim test or tail suspension test, though it was highly effective in littermate controls (FIGS. 2B-D). In the forced swim test, sleep deprivation reduced immobility time from 209.11±24.24 s to 47.85±8.94 s (p<0.001) in wild type mice while in A1R^{-/-} mice basal immobility (266.24±27.27 s), was not significantly affected by sleep deprivation (233.27±28.82 s; p=0.309). Confirming this result, sleep deprivation did not significantly reduce immobile time in A1R^{-/-} mice tested using the tail suspension test (A1R^{-/-}: 204.75±8.36 s; A1R^{-/-}+SD: 215.67±16.71 s; p=0.476).

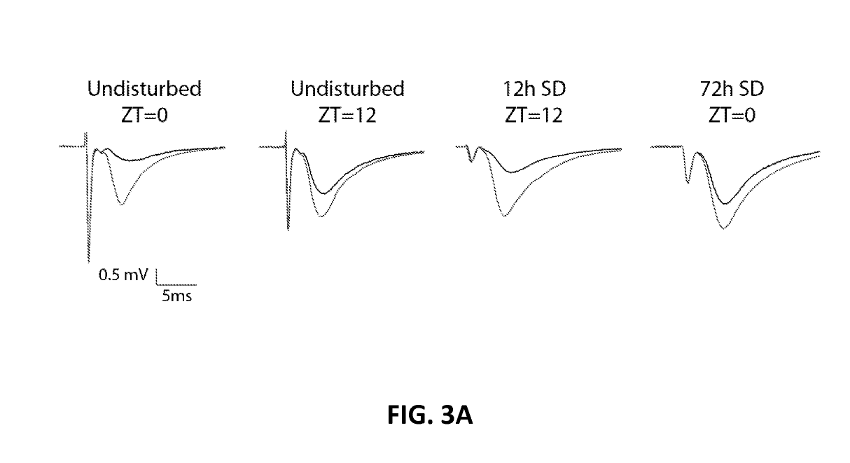

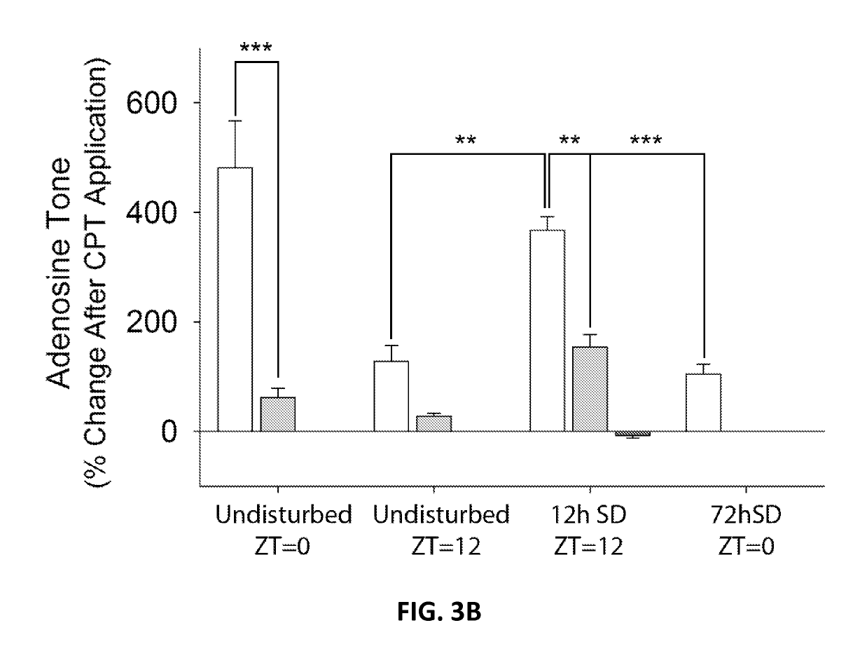

Sleep deprivation elevates astrocyte-derived adenosine. Astrocytic SNARE-dependent signaling activates neuronal A1 receptors. A relative measure of the extracellular adenosine level can be obtained by determining the proportional enhancement of synaptic transmission in response to the application of the A1R antagonist 8-cyclopentyltheophylline (CPT; 200 nM) an approach that has been validated using electrochemical biosensors (Schmitt, L. et al., J. Neurosci., 32:4417-25, 2012). At Zeitgeber time 0 (ZT=0), which corresponds with the time of lights on (onset of subjective night time), CPT caused a 481.70±85.04% increase in field excitatory postsynaptic potential fEPSP whereas at the end of the light phase (ZT=12) the proportional enhancement of fEPSP declined to 128.15±28.91% consistent with a light phase and sleep dependent reduction in adenosine tone (FIG. 3). This adenosine tone is regulated from an astrocytic source because conditional expression of dnSNARE significantly reduced the adenosine tone (ZT=0: 61.98±16.80%; ZT=12: 27.91±5.10%; p<0.001). Transient total sleep deprivation performed between ZT 0 and ZT 12 prevented the normal diurnal decline in adenosine tone (367.61±24.95%; p=0.028).

Although 12 h of sleep deprivation promoted antidepressive-like behavioral responses, 72 h of sustained sleep deprivation was ineffective. The inability of sustained sleep deprivation to promote antidepressive-like actions was paralleled by an inability to elevate adenosine tone (FIG. 3). These data demonstrate a significant correlation between the ability of sleep deprivation to both activate A1R signaling pathways and to promote antidepressive effects: 12 hours but not 72 hours of sleep deprivation leads to elevated A1R signaling as well as antidepressive-like actions.

Adenosine A1Rs are required for antidepressive effects of sleep deprivation. A1R^{-/-} mice do not express A1R mRNA transcripts, do not show immunoreactivity for A1R (FIG. 2A), do not respond to A1R agonists, but are viable and without gross anatomical abnormalities. Sleep deprivation failed to reduce the immobility time of A1R^{-/-} mice (FIG. 2B-D). In the forced swim test, sleep deprivation reduced immobility time from 144.95±21.74 s to 42.47±7.51 s (p<0.001) in wildtype mice while in A1R^{-/-} mice basal immobility (173.50±23.74 s), was not significantly affected by sleep deprivation (158.72±27.80 s; p=0.385). Similarly sleep deprivation did not significantly reduce immobile time in A1R^{-/-} mice tested using the tail suspension test (A1R^{-/-}: 204.75±8.36 s; A1R^{-/-}+sleep deprivation: 215.67±16.71 s; p=0.476).

Because the loss of A1R in the A1R^{-/-} mouse strain is not brain specific, the specific nervous system effects were tested for their role in mediating the effects of sleep deprivation. To achieve this objective, the A1R-specific antagonist, cyclopentyltheophylline (CPT; 4 mM), was introduced via intracerebroventricular (i.c.v.) administration. Neither i.c.v. implantation, vehicle infusion, nor CPT itself influenced forced swim task (FIG. 2B-D). Mice treated with i.c.v. CPT did not demonstrate the beneficial effects of sleep deprivation on immobility (FIG. 2B-D). In the forced swim test sleep deprivation reduced immobility time from 144.95±21.74 s to 42.47±7.51 s (p<0.001) in wildtype mice. Treatment with CPT did not affect basal immobility (176.43±21.77 s), but prevented sleep deprivation from reducing immobility in the forced swim (167.70±18.89 s; p=0.429) and tail suspension tests (CPT, basal immobility 187.60±11.46 s, sleep deprivation 204.00±3.58 s; p=0.318). Taken together these data show that astrocytic modulation of neuronal A1R mediates the beneficial effects of sleep deprivation on depressive-like symptoms.

The physiological mechanisms underlying the effects of sleep deprivation was investigated by asking whether sleep deprivation leads to an elevation of extracellular adenosine. A relative measure of the extracellular adenosine level can be obtained by determining the proportional enhancement of synaptic transmission in response to the application of the A1R antagonist 8-cyclopentyltheophylline (CPT; 200 nM). Independent measurements of extracellular adenosine using electrochemical biosensors have validated this approach. Field excitatory postsynaptic potentials (fEPSP) were measured at the Schaeffer collateral synapse of hippocampal area CA1 before and following application of CPT. At Zeitgeber time 0 (ZT=0), which corresponds to the time of lights on (subjective night time), addition of CPT causes a 481.70±85.04% increase in fEPSP, whereas at the end of the light phase (ZT=12) the proportional enhancement of fEPSP has declined to 128.15±28.91% consistent with a light phase and sleep dependent reduction in adenosine tone. This adenosine tone is regulated from an astrocytic source because conditional expression of dnSNARE in these glia significantly reduced the adenosine tone (ZT=0: 61.98±16.80%; ZT=12: 27.91±5.10%). Transient total sleep deprivation performed between ZT 0 and ZT 12 prevented the diurnal decline in adenosine tone (367.61±24.95%). In contrast, adenosine tone declined during sustained sleep deprivation (72 hours). At ZT=0 the adenosine tone is 481.70±85.04% in undisturbed mice whereas it has declined to 104.62±18.31% following 72 h of sleep deprivation (ZT=0). These data demonstrate that short term, but not extended sleep deprivation maintains wakefulness-dependent astrocyte-derived extracellular adenosine.

To investigate whether A1R activation alone is capable of altering the sleep architecture and mimicking the effects of sleep deprivation, the adenosine receptor agonist 2-chloro-N(6)-cyclopentyladenosine (CCPA; 500 nm) was administered via i.c.v. cannulae. Following recovery and one week of habituation to the headstage, EEG activity was recorded from mice with i.c.v. saline vehicle infusion for two days to establish baseline EEG activity patterns. The EEG changes following injection of the A1R agonist CCPA were monitored. CCPA resulted in a dramatic increase in the power spectra when compared to baseline saline power spectra (FIGS. 4A and 4B). The normalized power from 0.5 to 4.0 Hz was also analyzed, comparing saline baseline to CCPA infusion (FIG. 4D). The largest differences were seen in the first few hours of the administration of CCPA. The evaluation of individual vigilance states revealed a typical circadian distribution of the normal durations of wake (54.00±1.99%), REM sleep (9.30±1.10%), and non-REM sleep (35.57±1.99%) across the recording session during saline infusion (FIGS. 4C and 4D). In comparison, the duration of wake (31.38±1.93%), REM sleep (18.13±1.30%), and non-REM sleep (48.75±1.85%) changed significantly in this time frame with CCPA decreasing the amount of time that animals spent awake while increasing both REM and non-REM components.

To examine the potential efficacy of CCPA as a therapeutic agent in depression, CCPA (500 nm) was again administered through an i.c.v. cannulae. As determined using the forced swim test, CCPA decreased immobile time-demonstrating that it is effective at decreasing depression-like symptoms. To test the longevity of the beneficial effects of COPA administration, separate cohorts of mice were used. The cohorts were tested at 24 hours, +1 day, +3 days, and +5 days. Administration of COPA via three i.c.v. injections spaced evenly across a 12 hour period led to the observed beneficial effects indicated by the forced swim test last, which lasted for 24 hours. The beneficial effect on depression-like symptoms lasted an additional day, however 3 and 5 days after the COPA-infusion beneficial effects are no longer observed using the forced swim test. Using another test of depressive like symptoms, the sucrose consumption test, i.c.v. administration of COPA (51.45±6.35 g/kg) significantly increases saline baseline (30.55±3.60 g/kg) sucrose consumption (p=0.008), again demonstrating that adenosine agonists reduce depression-like symptoms.

Adenosine agonists cause a sustained enhancement of delta power during wakefulness. As a final step in testing the antidepressive actions of adenosine activation of A1Rs was determined to be sufficient to mimic the effects of sleep deprivation on depressive-like behaviors. An A1R agonist was delivered i.c.v. to cause a sustained (6-12 h) activation of the A1R (sleep deprivation studies showed that 12, but not 6 h of sleep deprivation is required for the antidepressive effects of forced wakefulness). The adenosine receptor agonist 2-chloro-N(6)-cyclopentyladenosine (CCPA; 500 nM) was delivered i.c.v. while performing EEG/EMG recordings. A single i.c.v. infusion of CCPA caused a transient decrease in the percentage of time that the animals were awake. Three consecutive CCPA infusions (ZT=0, 3, and 6) caused mice to sleep for a significantly greater time from ZT 1 through 10 compared to vehicle (FIG. 4A; p<0.002). By ZT 12, animals exhibited normal homecage and open field behaviors (FIG. 7A) and spent a significantly greater percentage of time awake than during vehicle administration or at 36 hours following administration (FIG. 7B).

Once mice awoke from the somnogenic effects of CCPA, there was a prominent increase in delta power (0.5-4 Hz.; p<0.001) during wakefulness that was sustained for 36 hours (p<0.001) following the onset of CCPA administration (FIGS. 4B-D). This increase in delta power is probably due to a long-lasting change in brain function, rather than due to the lingering presence of CCPA because i) the acute effect of CCPA was to reduce delta power, ii) effects of CCPA on sleep had reversed by ZT=11, which is 5 hours following the last administration of CCPA (at ZT=6).

Activation of A1R leads to sustained antidepressive-like behaviors. CCPA administration (ZT=0, 3, and 6) caused a reduction in immobile time when mice were tested at ZT=12 in the forced swim test (FIGS. 5A and 5B) without accompanying changes in locomotor activity assessed in both the open field and the rotorod (FIGS. 7A and 7C). Due to the complicated nature of using animal models of depression, an independent test was used to validate this result. Sucrose consumption which models anhedonia, a component of depression symptoms that is often incorporated in the evaluation for clinical diagnosis (2025), was used (El Yacoubi, M. et al., Proc. Natl. Acad. Sci. USA, 100:6227-32, 2003). Administration of CCPA (51.45±6.35 g/kg) significantly increased sucrose consumption compared to levels measured during the vehicle baseline (30.55±3.60 g/kg; p=0.008; FIG. 5C). Thus, in addition to the symptoms of despair or hopelessness modeled by the forced swim test, COPA also has beneficial effects on the lack of interest in pleasure modeled by the sucrose consumption test.

To test the longevity of the beneficial effects of COPA administration, separate cohorts of mice were tested at 12 hours (ZT=12), 36 hours, 84 hours, and 108 hours after first COPA treatment. The beneficial effects of only one subjective night of COPA treatment on immobility measured in the forced swim test last for up to 36 hours (FIG. 5B). Thus COPA can have rapid and moderately long lasting effects on depressive-like symptoms modeled in mice, which correspond with the duration of change in EEG delta power observed following COPA administration (FIGS. 4B-D, and FIG. 8).

### Example 2

Receptors are identified that cause an astrocyte-derived elevation of extracellular adenosine. The identified receptors, when activated, augment extracellular adenosine derived from an astrocytic source. Adenosine biosensors are used in frontal cortical slices and screen agonists for 10 receptors expressed in astrocytes. Slices from transgenic mice that selectively express dnSNARE only in astrocytes are used to determine whether receptor-induced adenosine accumulation is derived from an astrocytic source. Biosensor measurements are also performed in vivo to determine whether i.c.v. delivery of ligands elevates adenosine in the frontal cortex.

Sleep deprivation causes the accumulation of extracellular adenosine that is derived from an astrocytic source. Behavioral studies show that this sleep deprivation-dependent adenosine accumulation causes an A1R-dependent reduction in depressive-like behaviors. For example, sleep deprivation causes a reduction in immobility in the forced swim and tail suspension tests of behavioral despair (EXAMPLE 1). Evidence that this is mediated by an astrocytic source of adenosine is provided by the observation that the expression of dnSNARE selectively in astrocytes prevents both sleep deprivation-dependent increase in adenosine and the antidepressive-like effects of this behavioral manipulation (EXAMPLE 1). The importance of adenosine is confirmed by the fact that A1R^{-/-} mice and wildtype mice to which A1R antagonist has been delivered i.c.v. fail to respond to sleep deprivation with antidepressive-like responses (EXAMPLE 1). Finally, the concept that adenosine and A1R activation is sufficient to induce antidepressive actions is confirmed by the observation that i.c.v. infusion of A1R agonist leads to reduced immobility in the behavioral despair tasks and enhances sucrose consumption in a test of anhedonia (EXAMPLE 1). These studies place astrocyte-derived adenosine at the center of a novel strategy for antidepressant treatments. Since adenosine is used by cells throughout the body, however, it would be impossible to use adenosine therapeutics to treat depression. Instead, described herein is the identification of astrocytic receptors that cause the accumulation of adenosine within the CNS. Small molecules are then identified and used to activate the receptor, raise adenosine and alleviate depression and depressive-like symptoms.

Receptors and activity modulators are screened, starting with an initial ten likely candidate receptors (Table 1), for their ability to elevate adenosine. Identified receptors are then examined to determine if it/they act through an astrocytic mechanism that is essential for antidepressive-like behaviors. Finally, identified receptor activity is confirmed in vivo for the ability to elevate adenosine levels. This is performed using surgically implanted cannulae that allow for the direct delivery of candidate agonists in vivo. Identified receptors and agonists that cause an astrocyte-dependent accumulation of central adenosine are then used to develop blood brain penetrant compounds (EXAMPLE 3).

TABLE 1
Candidate receptors for stimulating adenosine release.

| Receptor | Agonist | Antagonist |
|---|---|---|
| AR1 | CCPA | CPT |
| PAR1 | TFLLR (SEQ ID NO: 1) | SCH79797 |
| P2Y14 | UDP glucose | UDP |
| Endothelin receptor B | BQ-3020 and IRL 1620 | BQ-788 and A192621 |
| Ntsr2 | Levocabastine and NT79 | |
| Mglur3 | Spaglumic acid, | LY 341495 |
| | L-CCG-I | |
| Mglur8 | RS-PPG | CPPG |
| Mglur5 | CHPG, DHPG | MPEP, MTEP |
| HR1 | 2-Pyridylethylamine | Ketotifen fumarate |
| | dihydrochloride | |
| P2Y1 | MRS2365 | MRS2500 |

Although the ten candidate receptors of Table 1 have not been observed to produce anti-depressive effects when activated, it is shown herein that it is necessary to perform sustained (6-12 hours) sleep deprivation and sustained activation of A1R during the subjective nighttime to lead to these antidepressive effects. Other studies have not used the temporal control necessary to reveal these effects. The initial candidate receptors and their respective agonists are selected from a list of 191 receptors identified in microarray data as being expressed in astrocytes. Some of these receptors are also known to mobilize Ca²⁺ in glia; for example, activation of PAR1 and P2Y1 receptors elevate astrocytic Ca²⁺ and preliminary studies show that PAR1 activation causes astrocytic-dependent dnSNARE sensitive accumulation of adenosine. Additionally, neurotensin receptor 2 is highly and selectively expressed in astrocytes (Furuta, A. et al., Brain Nerve, 59, 717-724, 2007).

Experiment 1:

Identify receptor agonists that stimulate an astrocyte-dependent elevation of extracellular adenosine in situ. Adenosine biosensors are gently inserted into frontal cortical slices and allowed to equilibrate for 30 minutes prior to taking a baseline recording of resting adenosine level. The brain slice is then superfused with candidate receptor agonists at a concentration equivalent to their EC₅₀ to identify those agonists that raise extracellular adenosine. Agonists that are identified to increase adenosine in the frontal cortex are then tested in brain slices obtained from mice in which dnSNARE is selectively expressed in astrocytes to prevent adenosine release from these glial cells. The result of these steps is the identification of ligand(s) that selectively activate glial-derived adenosine release.

It is important to control for tissue damage in these studies since cellular damage results in ATP leakage, leakage that is rapidly hydrolyzed to adenosine. To control for this possibility, brain slices isolated from dnSNARE mice are used. These mice allow for the determination that an astrocytic pathway is necessary for the adenosine accumulation and that accumulated adenosine does not result from damage.

Controls are also established to ensure determine that the biosensors are recording adenosine per se rather than downstream metabolites. Adenosine biosensors consist of platinum electrodes coated with three enzymes: adenosine deaminase, which converts adenosine to inosine; nucleoside phosphorylase, which converts inosine to hypoxanthine, and xanthine oxidase, which converts hypoxanthine to urate and H₂O₂. The H₂O₂ is then detected amperometrically. It is not possible to discriminate between adenosine and any of the resulting metabolites using a single electrode; thus after a first step in which agonists that cause an amperometric signal are identified using a mixed adenosine biosensor, recordings using pairs of biosensors in which the second sensor does not contain adenosine deaminase are then taken. This an inosine-sensing electrode (it will additionally sense hypoxanthine and urate). Simultaneous paired recordings are subtracted from adenosine and inosine biosensors to yield pure measures of adenosine. At the end of each brain slice experiment, biosensors are calibrated with adenosine and inosine to confirm selectivity of signals and to scale the resulting signals for subtraction.

A third control ensures that the agonists that are delivered do not stimulate signals on the biosensors and do not interfere with the enzyme activity that is required for the ability of the biosensor to detect adenosine. This is confirmed by performing in situ calibrations using the ligands and the biosensors. A fourth important control is to determine whether receptor antagonists prevent agonist-induced adenosine accumulation as this allows for the identification of the importance of a specific receptor in initiating the adenosine release pathway.

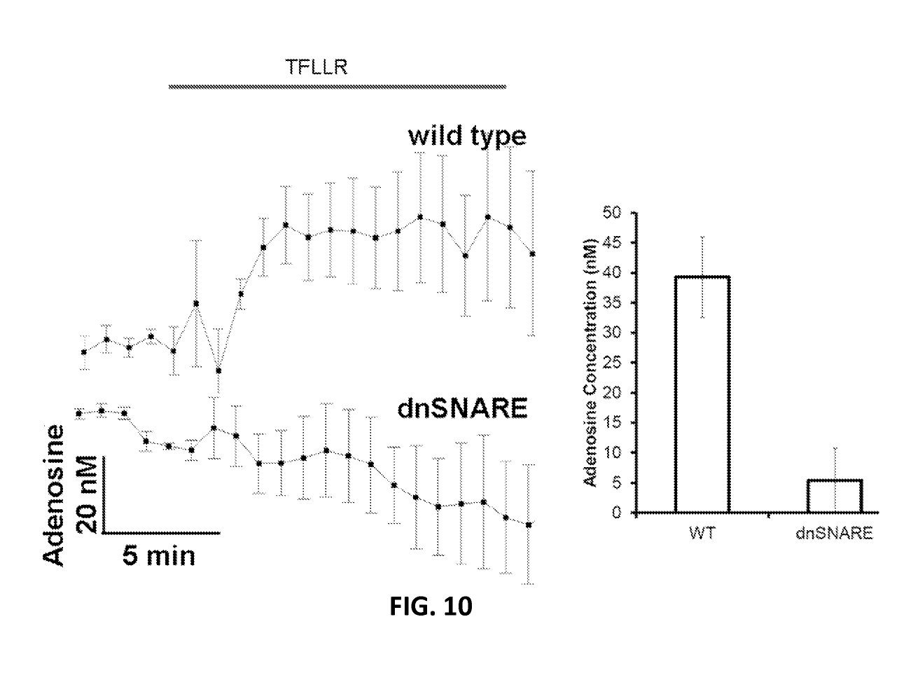

Experiments have shown that this screening approach is feasible and effective in identifying desired receptor agonists. Using the peptide ligand TFLLR (10-30 μM; SEQ ID NO:1), which activates PAR1, an astrocyte-dependent elevation of extracellular adenosine that is attenuated in dnSNARE slices is shown (FIG. 10). PAR1 elevates astrocytic Ca²⁺ and is preferentially expressed in these glia (Shigetomi, E. et al., J. Neurosci., 28:6659-6663, 2008). Given this positive result additional receptors and agonists can be found using the materials and methods described herein.

Experiment 2:

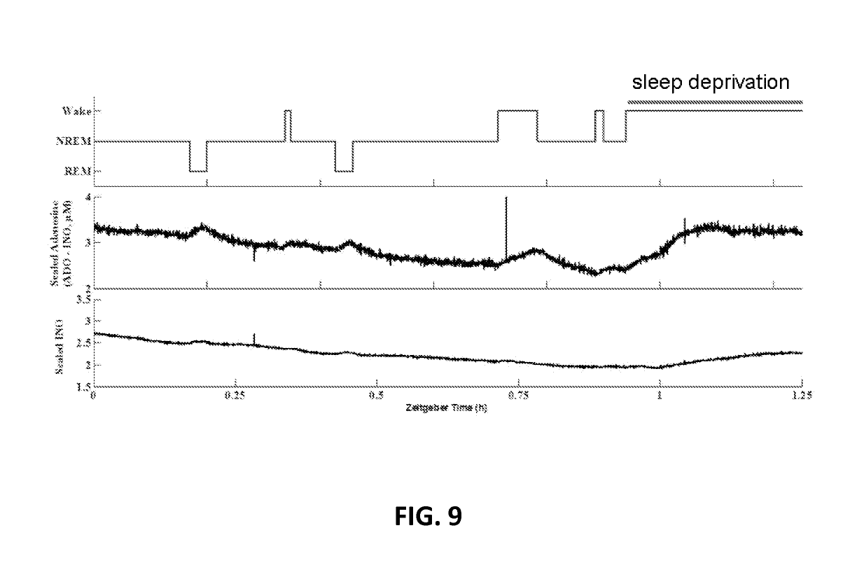

Determine whether delivery of receptor agonists in vivo elevates extracellular adenosine in freely behaving mice. Receptor agonists identified in Experiment 1 as stimulating an astrocyte-dependent elevation of extracellular adenosine are used to confirm that this group of compounds acts in a similar manner in vivo by performing amperometric measurements using paired biosensors (FIGS. 9 and 10).

Two cannulae are implanted into the mouse frontal cortex and one cannula for i.c.v. delivery of agonists. After one week for recovery adenosine and inosine biosensors are introduced into frontal cortex while implanting a dialysis probe for agonist delivery i.c.v. The agonist is infused during the light phase when adenosine levels are known to be low. Agonists are infused for a 30 minute period and adenosine levels quantified. After obtaining positive results, the experiments are repeated in dnSNARE mice to confirm that the resulting adenosine is derived from an astrocytic course and is not the result of tissue damage.

It is possible for some identified compounds and/or compounds yet to be identified that, when mice switch into wakefulness, either spontaneously or by gentle handling, extracellular adenosine rises rapidly (FIG. 9). As long as work is performed within the period of ZT=0 to ZT=6 spontaneous waking is brief (<500 seconds); thus with a 30 minute drug infusion period, such concerns are generally alleviated. If for certain compounds concerns remain, however, experiments are repeated using simultaneous EEG/EMG recording, a technique that has proven to be feasible.

The astrocyte is critical for mediating cellular and behavioral responses to sleep deprivation, and this glial cell selectively modulates slow-wave sleep (Halassa, M. et al., J. Neurosci., 276473-6477, 2007; Halassa, M. & Haydon, P., Annu. Rev. Physiol., 72:335-355, 2010). This glial pathway is required for antidepressive effects of sleep deprivation. Wakefulness stimulates the accumulation of adenosine from an astrocytic source. Having identified this novel, fast-acting antidepressive pathway all of the tools are in place (in situ and in vivo adenosine biosensors as well as tests of depressive-like behaviors) both to identify receptors that, when activated, cause the release of astrocyte-derived adenosine and to evaluate their potential for stimulating anti-depressive like effects.

### Example 3

Described herein are materials and methods to determine whether ligands that cause an astrocyte-derived elevation of adenosine lead to antidepressive effects. Data indicate that the antidepressive effects of wakefulness are mediated by a wakefulness-dependent elevation of adenosine acting on A1R. Pharmacological activation of A1R is similarly antidepressive even if mice are allowed to sleep. Receptor agonists that elevate adenosine (EXAMPLE 2) are delivered i.c.v. during the light phase when mice normally sleep and determine whether these agonists exert subsequent antidepressive actions. Three behavioral tests and their interactions with sleep deprivation are used to evaluate components of depressive behaviors: Porsolt swim test, tail suspension assay and the sucrose consumption test to model anhedonia (see EXAMPLE 1). Finally, dnSNARE are used mice to determine whether the antidepressive-like effects of receptor agonists require the astrocyte.

Receptors that stimulate astrocyte-dependent antidepressive actions are identified and developed chemical entities with the appropriate pharmacokinetic properties to induce antidepressive actions.

Described herein are materials and methods to determine whether ligands that cause an astrocyte-derived elevation of adenosine lead to antidepressive effects. The sub-group of agonists that elevate adenosine, as identified in EXAMPLE 2, are examined to determine if they are able to produce antidepressive-like actions.

All behavioral studies use C57Bl6/J mice, the genetic background of all mice discussed in this project, as this mouse has been shown to be ideal to study depressive-like behaviors (Cryan, J. et al., Trends Pharmacol. Sci., 23:238-245, 2002). These mice are evaluated using three tests—the forced swim test and tail suspension test, which measure behavioral despair, and sucrose consumption test, which measures anhedonia. These tests are sensitive to most antidepressive therapeutic agents and to sleep deprivation, a clinically relevant behavioral antidepressive stimulus.

Experiment 1:

Determine whether agonists that stimulate astrocyte-dependent adenosine accumulation lead to antidepressive-like actions. A guide cannula is implanted i.c.v. for drug delivery. One week after implantation, a specific receptor agonist, e.g., an agonist identified by the method of EXAMPLE 2, is introduced during the light phase to stimulate an elevation of astrocyte-derived adenosine. Subsequently at ZT=2, mice are subjected to the forced swim or tail suspension tests for behavioral despair. Individual mice are subjected to only one test. After identifying agonists that reduce immobility in these tests, experiments are repeated using dnSNARE mice to determine whether the antidepressant effect is mediated by an astrocytic pathway. It is important to note that the agonist is delivered during the light phase and animals are allowed to sleep. Animals are tested after drugs have been metabolized during the dark phase. This procedure allows one to dissociate immediate potential sleep-promoting effects of agonist delivery resulting from adenosine accumulation from delayed antidepressive actions observed during wakefulness.

An additional study using the sucrose consumption test is also used. This is a test that models anhedonia. After a period of habituation mice are given access to sucrose for three days to establish baseline sucrose consumption levels. They are subsequently given i.c.v. infusions of receptor agonists followed by the presentation of sucrose. The baseline sucrose consumption is then compared to the sucrose consumption following receptor agonist infusion.

In parallel experiments, immobility is determined in behavioral tests following i.c.v. administration of vehicle as an important control for statistical comparison. In addition to performing behavioral despair tests, locomotor behavior is examined in the open field test to confirm that reduced immobility in the forced swim and tail suspension tests does not result from a generalized stimulatory effect of the agonist. In studies performed using sleep deprivation and the A1R agonist CCPA delivered i.c.v. in the manner described herein, antidepressive-like effects were not due to generalized stimulation.

It is necessary to activate A1R for a sustained period of between 6 and 12 hours while mice sleep to produce antidepressive effects. Transient activation of A1R during the light phase was ineffective in producing antidepressive-like effects. To overcome this problem, repeated bolus injections of CCPA at ZT=0, 3 and 6 hours were used. This strategy we produced clear antidepressive effects in behavioral despair tasks as well as in the sucrose consumption test of anhedonia.

Experiment 2:

Determine the duration of anti-depressive effects of receptor agonists. Receptor agonists that have been identified to induce antidepressive-like effects are used to determine how long this action is sustained by treating mice with the agonists (i.c.v. at ZT=0, 3 and 6) and then take individual mice and subjecting them to the behavioral despair tasks at intervals of 12, 36, 84, and 108 hours following onset of drug delivery. Importantly, mice are tested only once.

Having identified receptors that activate the release of adenosine from astrocytes and that cause anti-depressive like actions, novel chemical entities are created with pharmaceutically desirable properties, as described herein, that allow for the effective delivery of agonists identified herein to a patient in need thereof. One of skill in the art would be able to use the methods described herein to identify such agonists, to use identify pharmaceutically effective amounts of such agonists, and to include such agonists in a formulation or composition suitable for effective pharmaceutical delivery to a patient suffering from depression or depressive-like symptoms. Both agonists and positive allosteric modulators are developed, which are, for example, screened in cell-based assays for efficacy and to identify optimal compounds and doses. Such compounds are used in pharmacokinetic studies to determine their ability, for example, to cross the blood brain barrier.

### Other Embodiments

Other embodiments will be evident to those of skill in the art. It should be understood that the foregoing detailed description is provided for clarity only and is merely exemplary. The spirit and scope of the present disclosure are not limited to the above examples, but are encompassed by the following claims. The contents of all references cited herein are incorporated by reference in their entireties.

1. A method of identifying a ligand that stimulates the cellular release of adenosine, comprising,
a) introducing a test compound into a subject, tissue sample or cultured cells; and
b) determining the release of adenosine,
wherein an increase in released adenosine is indicative of the test compound's efficacy as a ligand that stimulates the cellular release of adenosine.

2. The method of claim 1, wherein the extracellular adenosine concentration is determined using biosensor electrodes.

3. The method of claim 1, wherein the test compound is an activity modulator of a receptor expressed in astrocytes.

4. The method of claim 3, wherein the receptor and activity modulator are selected from the group of receptors and activity modulators of Table 1.

5. The method of claim 1, wherein the test compound is introduced into the frontal cortex of the subject or a brain slice from the frontal cortex of the subject.

6. The method of claim 1, further comprising introducing a test compound into a subject, tissue sample or cultured cells, wherein dnSNARE is selectively expressed in astrocytes of the subject, tissue sample or cultured cells.

7. A method of treating or preventing a disease, disorder or condition characterized by pathological sleep perturbations, comprising administering to a subject a therapeutically effective amount of a compound identified as a ligand that stimulates the cellular release of adenosine by the method of claim 1.

8. The method of claim 7, wherein the disease, disorder or condition is selected from the group consisting of: depression or depressive-like symptoms, sleep disorders in the elderly, Parkinson's disease, Alzheimer's disease, epilepsy, schizophrenia and symptoms experienced by recovering alcoholics.

9. A method of identifying a ligand that stimulates the cellular release of adenosine, comprising,
a) contacting cultured astrocytes or an astrocyte-based cell line with a test compound; and
b) determining the intracellular concentration of Ca²⁺,
wherein an increase in intracellular Ca²⁺ is indicative of the test compound's efficacy as a ligand that stimulates the cellular release of adenosine.

10. The method of claim 9, further comprising introducing the test compound into astrocytes of an animal model, subject or tissue sample and determining the concentration of extracellular adenosine, wherein an increase in extracellular adenosine validates the efficacy of the test compound as a ligand that stimulates the release of adenosine.

11. The method of claim 9, wherein the Ca²⁺ concentration is determined using a molecular marker indicative of Ca²⁺ concentration.

12. The method of claim 11, wherein the molecular marker is a fluorescent marker.

13. The method of claim 10, wherein the extracellular adenosine concentration is determined using biosensor electrodes.

14. The method of claim 9, wherein the astrocyte-based cell line is a human astrocytoma cell line.

15. A method of treating or preventing a disease, disorder or condition characterized by pathological sleep perturbations, comprising administering to a subject a therapeutically effective amount of a compound identified as a ligand that stimulates the cellular release of adenosine by the method of claim 9.

16. The method of claim 15, wherein the disease, disorder or condition is selected from the group consisting of: depression or depressive-like symptoms, sleep disorders in the elderly, Parkinson's disease, Alzheimer's disease, epilepsy, schizophrenia and symptoms experienced by recovering alcoholics.

17. A method of treating or preventing depression comprising administering an effective amount of an adenosine receptor agonist to a subject.

18. (canceled)

19. (canceled)

20. The method of claim 17, wherein the adenosine receptor agonist is a compound of formula I, 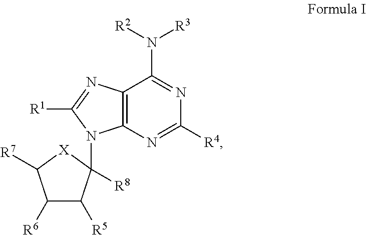
prodrugs, esters, or salts thereof wherein,
X is O, S, NH, or CH₂;
R¹, R², R³, R⁴, R⁵, R⁶, R⁷ and R⁸ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein each R¹, R², R³, R⁴, R⁵, R⁶, R⁷ and R⁸ are optionally substituted with one or more, the same or different, R⁹;
R⁹ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁹ is optionally substituted with one or more, the same or different, R¹⁰; and
R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

21.-30. (canceled)

31. The method of claim 17, wherein the adenosine receptor agonist is a compound of formula II, 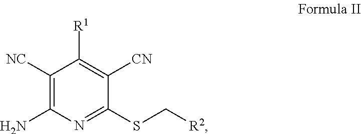
prodrugs, esters, and salts thereof wherein,
R¹ and R² are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein each R¹ and R² are optionally substituted with one or more, the same or different, R⁸;
R⁸ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁸ is optionally substituted with one or more, the same or different, R⁹;
R⁹ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁹ is optionally substituted with one or more, the same or different, R¹⁰; and
R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

32. (canceled)

33. (canceled)

34. The method of claim 31, wherein formula II has formula IIA, 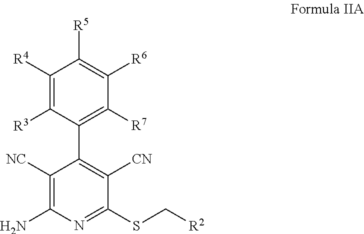
prodrugs, esters, or salts thereof wherein,
R² is a heterocyclyl optionally substituted with one or more, the same or different, R⁸;
R³, R⁴, R⁵, R⁶ and R⁷ are each the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein each R³, R⁴, R⁵, R⁶ and R⁷ are optionally substituted with one or more, the same or different, R⁹;
R⁸ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁸ is optionally substituted with one or more, the same or different, R⁹;
R⁹ is alkyl, halogen, nitro, cyano, hydroxy, amino, mercapto, formyl, carboxy, alkanoyl, carbamoyl, alkoxy, alkylthio, alkylamino, (alkyl)₂-amino, alkylsulfinyl, alkylsulfonyl, arylsulfonyl, carbocyclyl, aryl, or heterocyclyl, wherein R⁹ is optionally substituted with one or more, the same or different, R¹⁰; and
R¹⁰ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

35.-56. (canceled)
